SEQUENCE LISTING (6 sequences)

SEQ ID NO: 1 - DNA, 21 bp, Artificial Sequence
  Synthetic DNA Fragment
ttgactcttaagattatgtaa

SEQ ID NO: 2 - DNA, 21 bp, Artificial Sequence
  Synthetic DNA Fragment
tgggatggtcttagaatattt

SEQ ID NO: 3 - DNA, 82 bp, Artificial Sequence
  Synthetic DNA Fragment
gcctgcttttatctttgaacatattattaagcgaatgaagcctagtataatgaaaagcctaatggaccacaccattcctgag

SEQ ID NO: 4 - DNA, 82 bp, Artificial Sequence
  Synthetic DNA Fragment
ctcaggaatggtgtggtccattaggcttttcattatactaggcttcattcgcttaataatatgttcaaagataaaagcaggc

SEQ ID NO: 5 - DNA, 22 bp, Artificial Sequence
  Synthetic DNA Fragment
ctgcaaaaatgtctctctatcc

SEQ ID NO: 6 - DNA, 24 bp, Artificial Sequence
  Synthetic DNA Fragment
ggtgccgtgaattttaaacctcag

(19) United States
(12) Patent Application Publication
(10) Pub. No.: US 2019/0017054 A1
(43) Pub. Date: Jan. 17, 2019

(54) METHOD OF MODULATING SURVIVAL AND STEMNESS OF CANCER STEM CELLS BY MDA-9/SYNTENIN (SDCBP)

(71) Applicants: Paul B. FISHER, Henrico, VA (US); Sarmishta TALUKDAR, Richmond, VA (US); Luni EMDAD, Richmond, VA (US); Swadesh K. DAS, Richmond, VA (US); VIRGINIA COMMONWEALTH UNIVERSITY, Richmond, VA (US)

(72) Inventors: Paul B. FISHER, Henrico, VA (US); Sarmistha TALUKDAR, Richmond, VA (US); Luni EMDAD, Richmond, VA (US); Swadesh K. DAS, Richmond, VA (US)

(21) Appl. No.: 16/068,230

(22) PCT Filed: Jan. 6, 2017
(86) PCT No.: PCT/US2017/012486

§ 371 Date: Jul. 5, 2018

Related U.S. Application Data
(60) Provisional application No. 62/276,025, filed on Jan. 7, 2016.

(51) Int. Cl. C12N 15/113; G01N 33/50; A61P 35/00; G01N 33/574

(52) U.S. Cl. CPC C12N 15/1138; G01N 33/5073; A61P 35/00; C12N 2310/20; C12N 2310/14; C12N 2310/122; G01N 33/57492

(57) ABSTRACT

This invention discloses a method of modulating the survival and stemness of cancer stem cells (CSCs) by modulating the expression of MDA-9/Syntenin (SDCBP), which regulates multiple stemness genes, and controls survival of CSCs by activating the pathways, including without limitation NOTCH1. In one embodiment, the stemness genes that can be regulated by modulating expression or activity of MDA-9/Syntenin (SDCBP) includes, but are not limited to, ALDH1A1, AXL, CD44, DDR1, ID1, ITGB1, c-myc, Nanog, NOTCH, Oct4/POU5F1, Sox2, and STAT3. The invention also discloses a method of decreasing/inhibiting CSCs's tumorigenicity and a method of increasing survival of a subject with cancer by suppression of mda-9. This invention provides a method of inhibiting the growth of a cancer, and a method of determining the metastatic or angiogenic potential of a cancer. This invention further provides a method of screening for a candidate compound that modulate the expression or activities of MDA-9/Syntenin (SDCBP).

### CROSS-REFERENCE TO RELATED APPLICATIONS

This application claims the benefit of U.S. Ser. No. 62/276,025, filed Jan. 7, 2016, the entire content of which is incorporated herein by reference into this application. This application also cites various publications, the entire contents of which are incorporated herein by reference into this application.

This invention was made at least in part with government support under R01 CA134721 (PBF) awarded by the National Institutes of Health. The United States Government has certain rights in the invention.

### FIELD OF THE INVENTION

This invention relates to a method of modulating the survival and stemness of cancer stem cells (CSCs) by modulating the expression of MDA-9/Syntenin (SDCBP), which regulates multiple stemness genes, and controls the survival of CSCs by activating the pathways, including without limitation NOTCH1. In one embodiment, the stemness genes that can be regulated by MDA-9/Syntenin (SDCBP) includes, but are not limited to, ALDH1A1, AXL, CD44, DDR1, ID1, ITGB1, c-myc, Nanog, NOTCH, Oct4/POU5F1, Sox2, and STAT3. This invention also discloses a method of decreasing/inhibiting CSCs's tumorigenicity by suppression of mda-9. This invention provides a method of inhibiting the growth of a cancer, and a method of determining the metastatic or angiogenic potential of a cancer. This invention also discloses a method of increasing survival of a subject with cancer by suppression of mda-9. This invention further provides a method of screening for a candidate compound that modulate the expression or activities of MDA-9/Syntenin (SDCBP).

### BACKGROUND OF THE INVENTION

Cancer is multifactor in its etiology and multistep in its evolution (1). Since discovery and initial characterization in 1994, research on cancer stem cells (CSCs) has intensified providing convincing evidence that CSCs are major contributors to cancer growth, progression and resistance to therapeutic intervention (2). The concept that cancer is comprised of nearly homogenous, ectopically growing cells has been replaced with a more complex heterogeneous model in which cancer cells have varied potential to metastasize, interact and regrow after therapy (relapse) (3, 4). Many human tumors are organized as cellular hierarchies that are initiated and maintained by subpopulations of self-renewing CSCs (5). These subpopulations of cancer cells displaying high tumorigenic potential have been isolated from cancer patients with varied tumor types and display stemness properties (3, 6, 7, 8). Current consensus is that tumors comprised of cells with stem-like characteristics portend a poorer prognosis, which have important clinical implications for cancer diagnosis and treatment (9). Presence of a high proportion of CSCs also permits stratification of patients into a high metastatic risk group and represents an important area of clinical investigation (3, 5).

The most common form of brain tumors in adults is glioblastoma multiforme (GBM), an aggressive cancer that causes high mortality and morbidity. GBM currently remains one of the most difficult cancers to treat, with less than a 5% 5-year survival rate, despite multi-modality therapies including surgery, radiation therapy, and chemotherapy (10). This is potentially due to a lack of well-defined understanding of the mechanism(s) underlying GBM's complex heterogeneity, plasticity and therapy resistance. Isolation of stem cells from different normal and cancer tissue has been facilitated by the identification of specific cell surface markers. Recently, two mutually exclusive glioma stem cells (GSC) subtypes: pro-neural and mesenchymal, were identified and characterized with distinct dysregulated signaling pathways (11). CD133/Prominin-1 is an established and broadly accepted pro-neural GBM stem cell marker (7) that is shared in common with other CSCs from melanoma, prostate, pancreatic, liver, colon, lung, and ovarian cancers (2, 12). Recently, the importance of CD44 as an additional marker of mesenchymal GBM stem cells (11), as well as prostate and breast CSCs (2, 12), has been recognized. In prostate, alpha2beta1 integrin expression is also considered as both a normal and cancer stem cell marker (13). In breast cancer, the CD44+CD24^{-/low} expressing subpopulations are now generally accepted as representing a clinically relevant CSC phenotype and the presence of CSCs are positively associated with high-grade carcinomas (2, 6).

In addition to cell surface markers, several pathways and molecules that are involved in the control of self-renewal and differentiation of CSCs and normal stem cells include STAT3, NOTCH, C-Myc, NANOG, OCT4, SOX2 and others (2, 14, 15). These regulators of stemness also influence tumorigenesis and tumor progression (16). NOTCH and STAT3 signaling play critical roles in stem cell fate determination. OCT4, SOX2, and NANOG are central transcriptional regulators of stemness, forming an interconnected autoregulatory network to maintain cell pluripotency and self-renewal (14). NOTCH1, SOX2, and CD133 are known to regulate the pro-neural glioma stem cells (GSC) subtype, whereas CD44 is believed to regulate the mesenchymal GSC subtype (11). Moreover, many aggressive cancers that result in poor patient survival show higher expression of these stemness genes (17, 18). Despite clinical significance, effective/selective targeting strategies for CSCs, including GSCs, do not currently exist (19).

MDA-9/Syntenin (SDCBP) is a scaffold protein that interacts with a remarkable repertoire of key regulatory proteins, including SRC, FAK and EGFR, which are often related to expression of the tumor phenotype and cancer progression (10, 20). MDA-9 is a diagnostic marker of tumor aggression and grade in gliomas (21), melanomas (22, 23), and breast cancer (24). Based on these observations, it was hypothesized that higher tumor grade, which correlates with a more invasive and metastatic phenotype, would consist of an increased proportion of CSCs that would express elevated levels of MDA-9. CSCs are major contributors to cancer progression (2) and MDA-9 plays a seminal role in the progression of several cancer types (10, 20-24). Accordingly, this invention currently assessed the association between stemness and MDA-9 expression in glioblastoma multiforme (GBM), prostate and breast cancer as well as in normal astrocytes, and normal prostate and breast epithelial cells. Stemness is defined as the ability of stem cells to self-renew and differentiate (25). This property was studied by using sphere formation assays, cell-surface based stem population assessment, monitoring genes regulating self-renewal, and tumorigenicity. The influence of MDA-9 on CSC survival, growth, angiogenesis and chemoresistance was also examined. Finally, this invention dissected the mechanisms contributing to MDA-9-mediated stem phenotypes and survival. This invention now demonstrates for the first time that MDA-9 promotes stem cell phenotypes and survival through regulation of NOTCH1, C-Myc, STAT3 and Nanog in GBM, prostate and breast CSCs.

### SUMMARY OF THE INVENTION

This invention discloses a method of modulating the survival and stemness of cancer stem cells (CSCs) by modulating the expression of MDA-9/Syntenin (SDCBP), which regulates multiple stemness genes, and controls survival of CSCs by activating the pathways, including without limitation NOTCH1. In one embodiment, the stemness genes that can be regulated include, but are not limited to, ALDH1A1, AXL, CD44, DDR1, ID1, ITGB1, Nanog, NOTCH, Oct4/POU5F1, Sox2, and STAT3. The invention also discloses a method of decreasing/inhibiting CSCs's tumorigenicity by suppression of mda-9. The invention also discloses a method of increasing survival of a subject with cancer by suppression of mda-9. The invention also provides a method of distinguishing a non-stem normal cell from a non-cancer stem cell from a cancer stem cell. This invention provides a method of inhibiting the growth of a cancer, and a method of determining the metastatic or angiogenic potential of a cancer. This invention further provides a method of screening for a candidate compound that modulates the expression or activities of MDA-9/Syntenin (SDCBP).

### BRIEF DESCRIPTION OF THE FIGURES

FIGS. 1A to 1G: mda-9 expression correlates with stemness markers in clinical samples and overexpression of mda-9 enhances stemness markers in normal stem cells and CSCs. FIG. 1A: clinical array data confirms a strong correlation between expression of mda-9 with the stemness markers Nanog and CD133. FIG. 1B: CSC array data demonstrates dramatic downregulation of stem cell markers in mda-9 knockdown (kd) CSCs. FIG. 1C is a graphical plot showing the expression and association of c-myc, Nanog, CD133 and mda-9 in different clinical samples (n=48). FIG. 1D is a self-renewal analysis in primary human astrocytes (HA), and mda-9 overexpressing HA stem cells (HA+mda-9), as well as in control (shcon) and mda-9 knockdown (shmda-9) stem cells from VG2, VG9 and U-1242 GBM cells. FIG. 1E: Left upper panel is a live image analysis of human primary astrocyte (HA) stem cell neurospheres; Left lower panel is a FACS analysis of stem cell (SC) markers in null vector- and mda-9-overexpressing HA neurospheres; Right upper panel shows mda-9 expression in HA stem cells as compared to U-1242 NSCCs and CSCs; Right lower panel shows that overexpression of mda-9 significantly enhances several stem cell markers in HA cells. FIG. 1F shows the FACS (left panel) and RT-PCR (right panel) analysis of stem cell (SC) markers and stemness genes in null vector- and mda-9-overexpressing VG2 non-stem cancer cells. FIG. 1G shows the FACS (left panel) and RT-PCR (right panel) analysis of stem cell (SC) markers and stemness genes in null vector- and mda-9-overexpressing U-1242 non-stem cancer cells. Relative expression indicates fold change in expression. Bars represent the standard error of the mean (SEM). See also Tables 1 to 3. *P<0.05, **P<0.01 using student t-test and ANOVA.

FIGS. 2A to 2F: Gain of function studies indicate that mda-9 expression correlates with stemness properties in normal stem cells and CSCs. FIG. 2A shows FACS analyses of stem markers in immortal normal primary human prostate epithelial stem cells (RWPE-1) and RWPE-1 overexpressing mda-9 (RWPE-1 mda-9) cells. The upper panel shows Phase contrast images of sphere formation in RWPE-1 and RWPE-1 mda-9 cells. The lower panel is a tabular compilation of expression of CD44, CD133 and integrin α2β1. FIG. 2B shows the live imaging analysis of self-renewal and spheroid size of RWPE-1 and RWPE-1 mda-9 cells over time. FIG. 2C shows the flow cytometry and RT-PCR analysis of CSC markers and gene expression in Ad.5/3. null and Ad.5/3. mda-9 overexpressing non-stem cancer cells (NSCC) from prostate cancers (DU-145) and breast cancers (MDA-MB-231). FIG. 2D shows the RT-PCR analysis of mda-9 in stem cells from normal prostate epithelial cells (RWPE-1), and stem/non-stem cancer cells from prostate cancer (DU-145). FIG. 2E shows the RT-PCR analysis of mda-9 and stem genes (Nanog, Oct4, CD44 and CD133) in stem/non-stem cells from normal astrocyte (HA). FIG. 2F shows the RT-PCR analysis of mda-9, stem genes (Nang and Oct4) and mda-9 downstream target genes (MIF and IGFBP2), in control and mda-9 overexpressing normal prostate cells (RWPE-1). Bars represent the standard error of the mean (SEM).

FIGS. 3A-3D show that mda-9 indirectly regulates STAT3 activity. FIG. 3A is the flow cytometry analysis of p-STAT3 in control and mda-9 kd CSCs from clinical GBM (VG2, VG9) and the GBM cell line U-1242. FIG. 3B is the RT-PCR analysis for expression of mda-9 and stemness genes in shcon, mda-9 kd, and mda-9 kd cells overexpressing constitutively active (CA) STAT3. Relative expression indicates fold change in expression.

FIG. 3C is the image analysis of shcon, mda-9 kd and mda-9 cells overexpressing constitutively active (CA) STAT3 or CA Src. FIG. 3D is the flow cytometry analysis of p44/42, phosphor-p44/42 and IGF1R in shcon and mda-9 kd CSCs from GBM clinical samples, DU-145 and MDA-MB-231 cell lines. *P<0.05, using student t-test and ANOVA. * indicates significance between shmda-9 and shmda-9+CA STAT3 groups.

FIG. 4. mda-9 regulates molecules and pathways associated with stemness and survival. Expression of the indicated proteins by Western blot analysis in control and mda-9 knockdown CSCs.

FIGS. 5A-5D. mda-9 regulates stem cell phenotypes through STAT3 and Src activation. FIG. 5A is the flow cytometry analysis of shcon and shmda-9 CSCs for p-STAT3 expression. FIG. 5B is the flow cytometry analysis of shcon and shmda-9 CSCs for p-Src expression. FIG. 5C is the flow cytometry analysis of shcon and shmda-9 CSCs for p44/42, and phospho-p44/42. FIG. 5D is the live image analysis of shcon and mda-9 kd CSCs overexpressing non-constitutively activated (NCA) Src and the scale bar is 100 μm.

FIGS. 6A to 6D. Suppression of mda-9 expression decreases CSC survival, tumorigenesis and metastasis. FIG. 6A is the live/dead fluorescent images and flow cytometry analyses in GBM clinical samples (VG2, VG9) and cell line (U-1242) which show an increased percentage of cell death and apoptosis caused by kd of mda-9. FIG. 6B is the live/dead fluorescent images and flow cytometry analyses in the prostate cancer cell line (DU-145) and the breast cancer cell line (MDA-MB-231) which demonstrate an increased percentage of cell death and apoptosis caused by kd of mda-9. FIG. 6C: upper panel shows the bioluminescent imaging (BLI) of intracranial GBM which indicates intense luciferase activities in shcon mice as compared to the mda-9 kd group; middle and lower panels respectively show BLI using mouse metastatic models of shcon and mda-9 kd prostate (ARCaP-M), and kd breast (MDA-MB-231) CSCs. FIG. 6D is the survival analysis of mice plotted over time showing the cumulative effect of mda-9 kd in GSCs. Knocking down mda-9 increased survival time (p=0.04, log rank test) relative to control. *p<0.05.

FIGS. 7A to 7C: mda-9 regulates CSC survival and growth. FIG. 7A is the flow cytometry analysis of cell viability in DU-145 and MDA-MB-231 control and mda-9 kd cells. FIG. 7B shows images of Hematoxylin and Eosin (H&E) staining of tissue collected from shcon and shmda-9 intracranial orthotopic brain tumors at 40, 100, and 400× magnification. FIG. 7C is the anchorage independent growth assay comparing colony formation of control and mda-9 kd CSCs. Bars represent SEM.

FIGS. 8A to 8C: knockdown (Kd) of mda-9 decreases tumorigenicity. FIG. 8A shows the tumor size and volume of control and mda-9 kd prostate (left) and breast (right) CSC subcutaneous xenografts in nude mice. FIG. 8B shows the flow cytometry analysis of control subcutaneous xenograft tumors to quantify expression of stem markers. FIG. 8C shows the tumor size and stem marker expression of CSC xenografts in nude mice injected intratumorally with Ad.5/3.shcon or Ad.5/3.shmda-9. Bars represent SEM.

FIGS. 9A to 9D: mda-9 regulates cell-matrix and cell-cell attachment in CSCs. FIG. 9A is the live image analysis of control and mda-9 kd GBM CSCs on 2D and 3D matrigel. FIGS. 9B and 9C are the live time-lapse imaging of control and mda-9 kd CSCs from DU-145 (FIG. 9B) and from MDA-MB-231 cells (FIG. 9C). FIG. 9D is the Phase contrast image analysis of 2D and 3D attachment of control and mda-9 kd DU-145 and MDA-MB-231 CSCs. The arrow shows cell spreading. Bars SEM.

FIGS. 10A and 10B: mda-9 regulates the NOTCH1 pathway by regulating NOTCH1 degradation and activation. FIG. 10A shows the flow cytometry analyses of control and mda-9 kd CSCs from GBM clinical samples and cell line for surface expression of NOTCH1 and DLL1. FIG. 10B shows the flow cytometry analyses of control and mda-9 KD CSCs from GBM clinical samples and cell lines for NUMB and p-SRC expression.

FIGS. 11 A to 11D: mda-9 regulates CSC survival by controlling c-myc through the NOTCH1 pathway. FIG. 11A is the flow cytometry analysis of shcon and mda-9 kd CSCs from prostate and breast cancer cell lines for NUMB expression. FIG. 11B is peptide blocking and recovery of function studies to elucidate the effect of NOTCH1 blocking peptide (NBP) and FIG. 11C is c-myc overexpression on shcon and mda-9 kd CSCs. FIG. 11D is c-myc expression in control, shmda-9 and c-myc overexpressing CSCs. Bars represent SEM.

FIGS. 12A to 12C: mda-9 regulates CSC survival by controlling c-myc through the NOTCH1/RBPJK pathway. FIG. 12A is the luciferase reporter assay analysis of control and mda-9 kd CSCs from GBM clinical samples (VG2 and VG9) and GBM cell line (U-1242) and prostate cancer (DU-145) and breast cancer (MDA-MB-231) cell lines for RBPJK promoter activity. FIG. 12B shows the RT-PCR-based c-myc expression in control and mda-9 kd CSCs from GBM cells. Relative expression indicates fold change in expression. FIG. 12C refers to the peptide blocking and recovery of function studies which elucidate the effect of Notch1 blocking peptide (NBP) and c-myc overexpression on shcon and mda-9 kd GBM CSCs, respectively. Bars represent SEM. *P<0.05, **P<0.01 using student t-test and ANOVA.

FIGS. 13A to 13D: mda-9 regulates apoptosis by p27/Kip-1 expression through the NOTCH1/RBPJK/c-Myc pathway. FIG. 13A is the RT-PCR analysis of p27/Kip-1 and miR-221 expression in shcon and mda-9 kd GBM CSCs. FIG. 13B is the RT-PCR analysis of p27/Kip-1, mda-9, and c-myc in shcon, mda-9 kd CSCs and mda-9 kd CSCs overexpressing c-myc. * indicates significance in expression of p27/kip-1 and c-myc between the shmda-9 and shmda-9+c-myc groups. Relative expression indicates fold change in expression. FIG. 13C shows the image analysis of control and p27 overexpressing CSCs. FIG. 13D shows caspase 3/7 activation analysis in shcon and shmda-9 CSCs. Bars represent SEM. *P<0.05, using student t-test and ANOVA.

FIGS. 14A to 14D. mda-9 regulates angiogenic potential in CSCs. FIG. 14A is the chorioallontoic membrane (CAM) chick embryo assay showing angiogenic potential of control, mda-9 overexpressing and mda-9 kd cells. FIGS. 14B and 14C are ELISA and protein array analysis of conditioned media from control and shmda-9 CSCs, respectively. Boxes show significant change in expression of angiogenic proteins. FIG. 14D shows OCT4 and SOX2 expression in control and shmda-9 CSCs. Bars represent SEM. See also Table 4.

FIG. 15 is the schematic representation of MDA-9-mediated regulation of CSC survival and stemness. MDA-9 regulates stem cell survival and pluripotency by regulating several molecular activities and promoting defined gene expression changes. The survival pathway is affected by the expression, degradation or activation of the constituents NOTCH1/C-Myc signaling pathway. Stemness is regulated by the STAT3/Nanog signaling pathway, which is likely regulated by p-p44/42 and IGF1R. Ub=Ubiquitin, PDL=PDZ ligand, ICD=Intracellular domain, DLL1=Delta-like protein 1, LNX1=Ligand of numb protein 1, RBPJK=Recombining binding protein suppressor of hairless.

### DETAILED DESCRIPTION OF THE INVENTION

This invention relates to a method of modulating the survival and stemness of cancer stem cells (CSCs) by modulating the expression of MDA-9/Syntenin (SDCBP), which regulates multiple stemness genes, and controls the survival of CSCs by activating the pathways, including without limitation NOTCH1. In one embodiment, the stemness genes that can be regulated by includes, but are not limited to, ALDH1A1, AXL, CD44, DDR1, ID1, ITGB1, c-myc, Nanog, NOTCH, Oct4/POU5F1, Sox2, and STAT3. This invention also discloses a method of decreasing/inhibiting CSCs's tumorigenicity by suppression of mda-9. This invention also discloses a method of increasing survival of a subject with cancer by suppression of mda-9. The invention also provides a method of distinguishing a non-stem normal cell from a non-cancer stem cell from a cancer stem cell. This invention provides a method of inhibiting the growth of a cancer, and a method of determining the metastatic or angiogenic potential of a cancer. This invention further provides a method of screening for a candidate compound that modulate the expression or activities of MDA-9/Syntenin (SDCBP).

In one embodiment, this invention provides a method of modulating the expression of one or more stemness regulators in cancer stem cells, the method comprises a step of modulating the expression of MDA-9/Syntenin (SDCBP) in said cancer stem cells. In one embodiment, the stemness regulator is a nucleic acid which regulates the self-renewal and/or pluripotency of the cancer stem cell. In another embodiment, the stemness regulators include, but are not limited to, ALDH1A1, AXL, CD44, DDR1, ID1, ITGB1, c-Myc, Nanog, NOTCH, Oct4/POU5F1, Sox2, and STATS.

In one embodiment of the present invention, the reduction in the expression of MDA-9/Syntenin (SDCBP) decreases the expression of Nanog, Oct4 and/or Sox2 through the regulation of the STAT3/Nanog pathway. In another embodiment, the reduction in the expression of MDA-9/Syntenin (SDCBP) decreases the expression of c-Myc through the regulation of the NOTCH1 pathway.

In one embodiment of the present invention, the apoptosis of the cancer stem cells is increased. In another embodiment, the apoptosis of the cancer stem cells is increased through the NOTCH1/RBPJK/C-Myc pathway or the cIAP2 pathway.

In one embodiment of the present invention, the stem cells come from a cancer includes, but is not limited to, prostate cancer, breast cancer, gastric cancer, lung cancer, brain cancer, pancreatic cancer and neuroblastoma.

In one embodiment of the present invention, the expression of MDA-9/Syntenin (SDCBP) is modulated with an agent, or with mutation, inactivation, knockdown or deletion of the gene of MDA-9/Syntenin (SDCBP). In one embodiment, the agent is a small interfering RNA (siRNA) or a short hairpin RNA (shRNA) comprising a sequence specific for the gene of MDA-9/Syntenin (SDCBP) or using CRSIPR/Cas9 or similar genome targeted editing approach. In another embodiment, the mutation, inactivation, knockdown or deletion of the gene of MDA-9/Syntenin (SDCBP) is achieved by CRSIPR/Cas9 or other genome targeted editing techniques.

In one embodiment, the survival of the cancer stem cells is controlled via activation of the NOTCH1 pathway through phospho-Src and DLL1.

In one embodiment, this inventions provides a method of testing a compound for its ability to modulate the expression or activities of MDA-9/Syntenin (SDCBP), the method comprises the steps of (i) contacting a population of cells with said compound; and (ii) determining the expression or activities of MDA-9/Syntenin (SDCBP) in said cells in the presence and absence of said compound, wherein a change in the expression or activities of MDA-9/Syntenin (SDCBP) in the presence of said compound as compared to the absence of said compound indicates that said compound is capable of modulating the expression or activities of MDA-9/Syntenin (SDCBP). In another embodiment, the population of cells are cancer stem cells or non-stem cancer cells.

In one embodiment, this inventions provides a method of inhibiting the growth of a cancer, the method comprises a step of inhibiting the expression of MDA-9/Syntenin (SDCBP) in the stem cells of said cancer. In another embodiment, the expression of MDA-9/Syntenin (SDCBP) is inhibited with an agent or with gene mutation, inactivation, knockdown or deletion. In one embodiment, apoptosis of the stem cells is increased, or the metastasis or angiogenesis of said cancer is inhibited.

In one embodiment, this inventions provides a method of determining the metastatic or angiogenic potential of a cancer, the method comprises a step of comparing the level of expression of MDA-9/Syntenin (SDCBP) in the stem cells of said cancer with that in non-cancer stem cells, wherein an increased level of expression indicates an increased potential for metastasis or angiogenesis of said cancer. In one embodiment, the stem cells come from a cancer includes, but is not limited to, prostate cancer, breast cancer, gastric cancer, lung cancer, brain cancer, pancreatic cancer and neuroblastoma.

With the discovery of the effects of the mda-9 gene on cancer cells, this invention provides methods of modulating the self-renewal, pluripotency, apoptosis and/or survival of cancer stem cells or non-stem cancer cells through the inhibition of mda-9. The effects of mda-9 can be altered by modulating the expression of the MDA-9/Syntenin (SDCBP) gene, or the activities of the MDA-9/Syntenin (SDCBP) protein. In one embodiment, the transformation-associated effects of mda-9 is inhibited genetically by inhibiting/inactivating the mda-9 gene using shRNA, siRNA, and the like, or by knocking-out/deleting the mda-9 gene using CRISPR/cas9 or other genome targeted editing techniques. In another embodiment, the transformation-associated effects of mda-9 is inhibited pharmacologically by blocking the ability of MDA-9 protein to interact with its partner proteins such as src, EGFR and IGF1R.

In one embodiment, this invention provides a method of distinguishing between normal stem cells from non-stem cancer cells from cancer stem cells by monitoring the level of mda-9 RNA and/or the MDA-9 protein in applicable tissues or cell component (e.g. body fluids and exosomes). In another embodiment, this invention provides a method of monitoring or determining the metastatic potential of a cancer. By monitoring the level of mda-9 RNA and/or the MDA-9 protein in the cells such as the circulating tumor cells, it is possible to assess the aggressiveness of the cancer cells and thereby determining the metastatic potential of the cancer.

The present data suggests that mda-9 has a role as a regulator of tumor cells and cancer stem cell angiogenesis. In one embodiment, this invention provides a method of regulating angiogenesis of a cancer stem cell through the alteration of gene expression of one or more genes by modulating the expression of mda-9 gene (genetically or pharmacologically) or activity of the MDA-9 protein. In one embodiment, the inhibition of the mda-9 gene alters the expression level of genes listed in Table 4. These genes include but are not limited to angiogenin, angiopoietin, CXCL16, GM-CSF, IGFBP2, and IL-8, and are present in at least prostate cancer and breast cancer cells.

In one embodiment, this invention provides a method of testing a compound that can modulate the expression or activities of MDA-9/Syntenin (SDCBP) by treating a population of cells with a candidate compound and determining the expression or activities of MDA-9/Syntenin (SDCBP) in said cells in the presence and absence of said compound. Various bioassays and biochemical/molecular assays that can measure or monitor the expression or activities of MDA-9/Syntenin (SDCBP) can be used in the present invention. In one embodiment, the assays include but are not limited to, invasion assay, western blotting (for evaluating downstream genes regulated by MDA-9/Syntenin (SDCBP)), measurement of changes in phosphorylation of target molecules (such as src or EGFR), and measurement of changes in secretion of target proteins by cancer cells (such as IGFBP2).

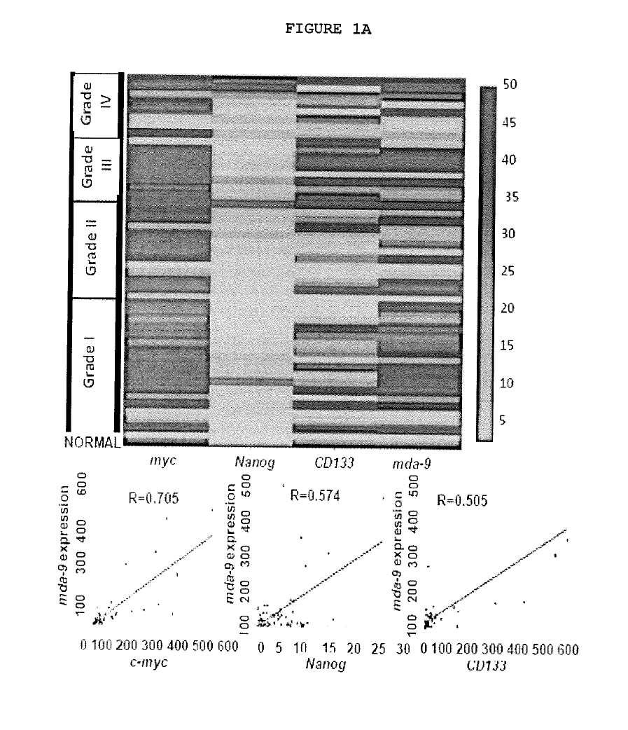

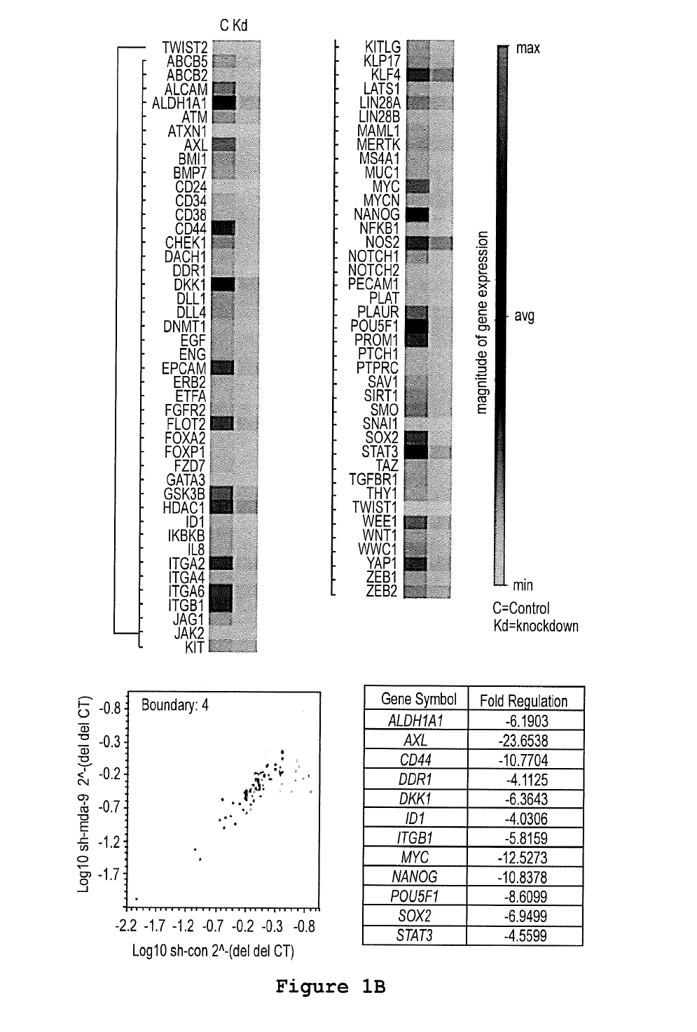

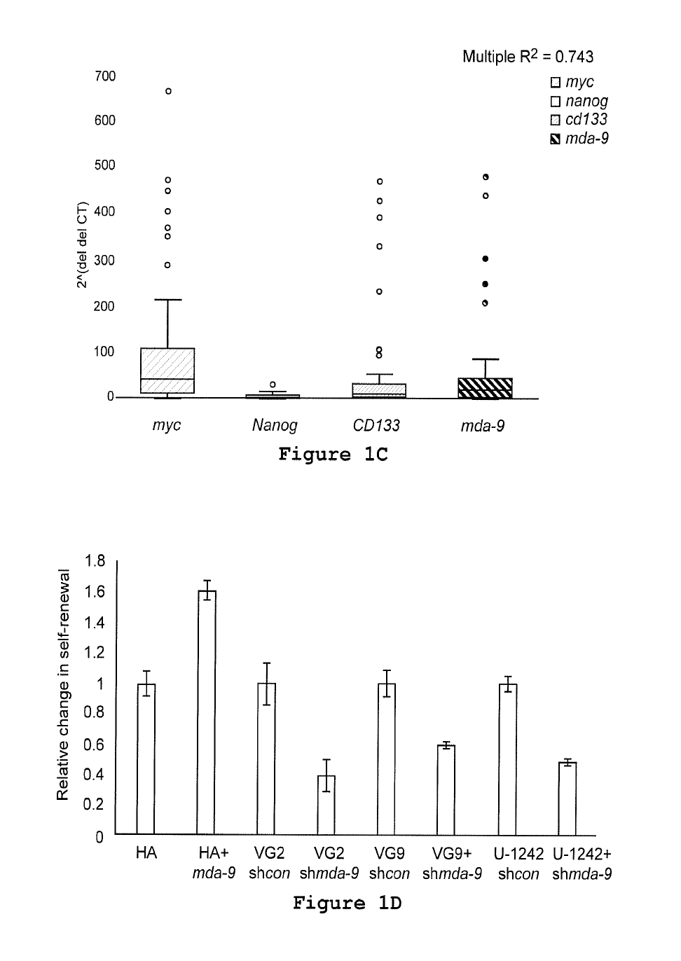

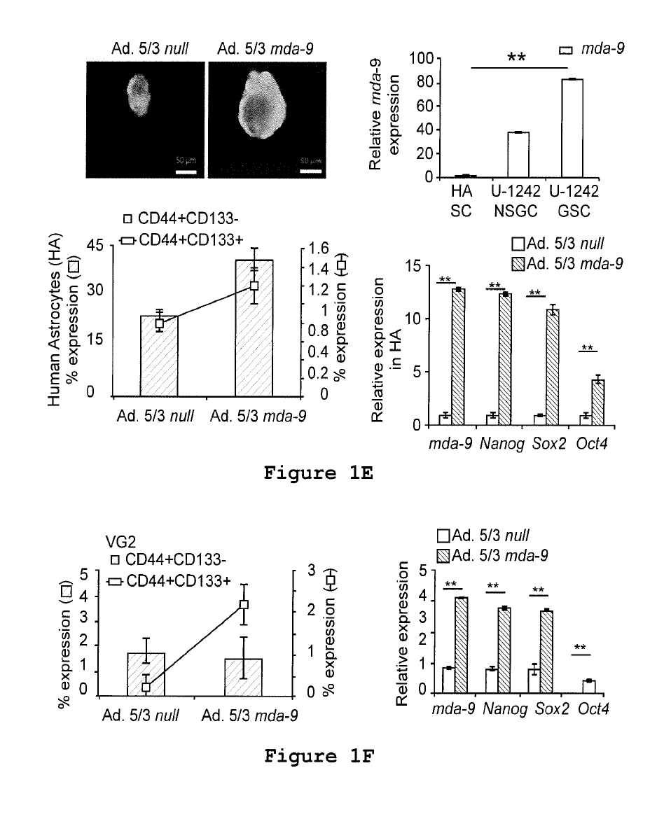

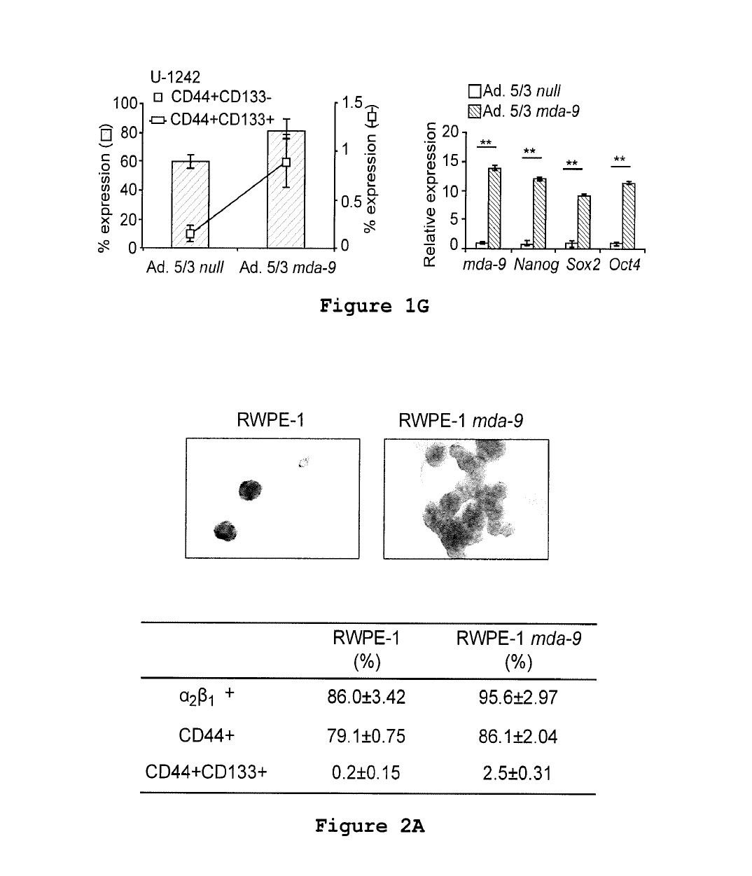

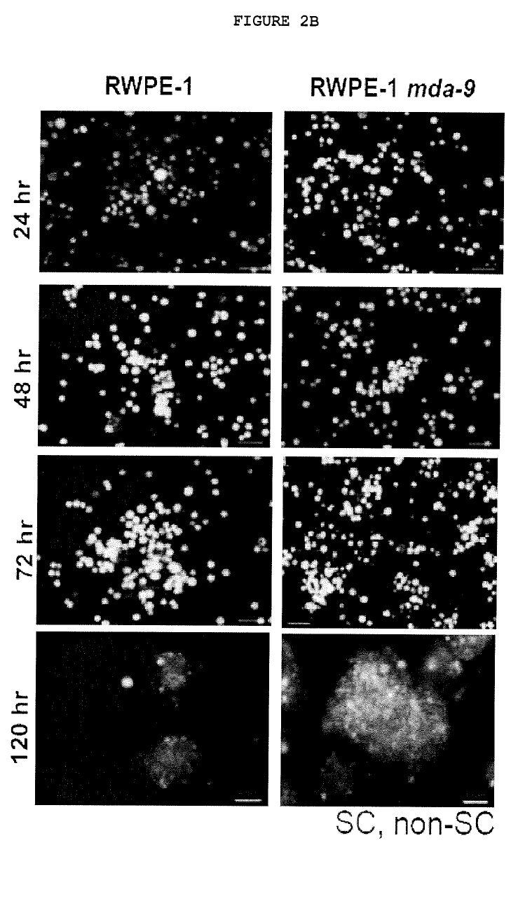

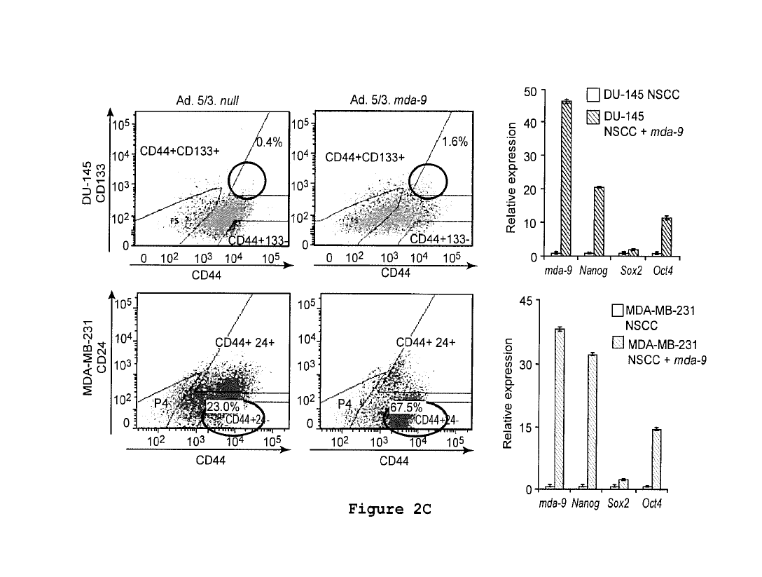

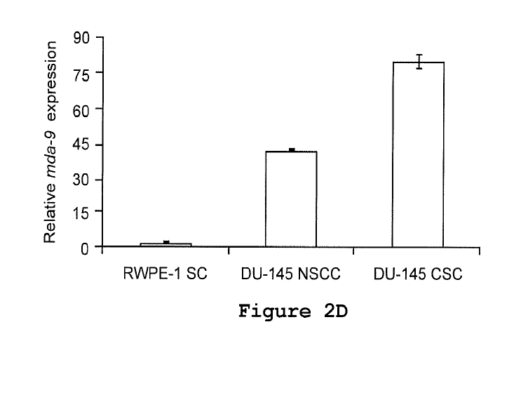

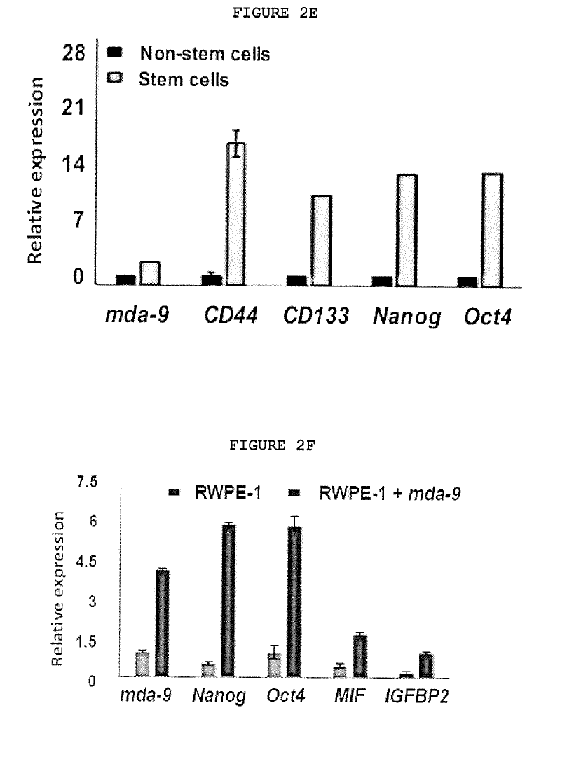

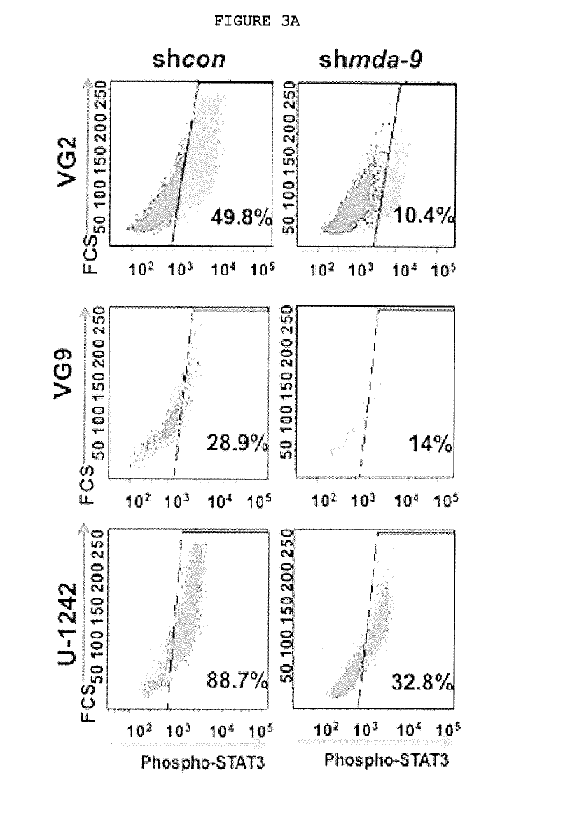

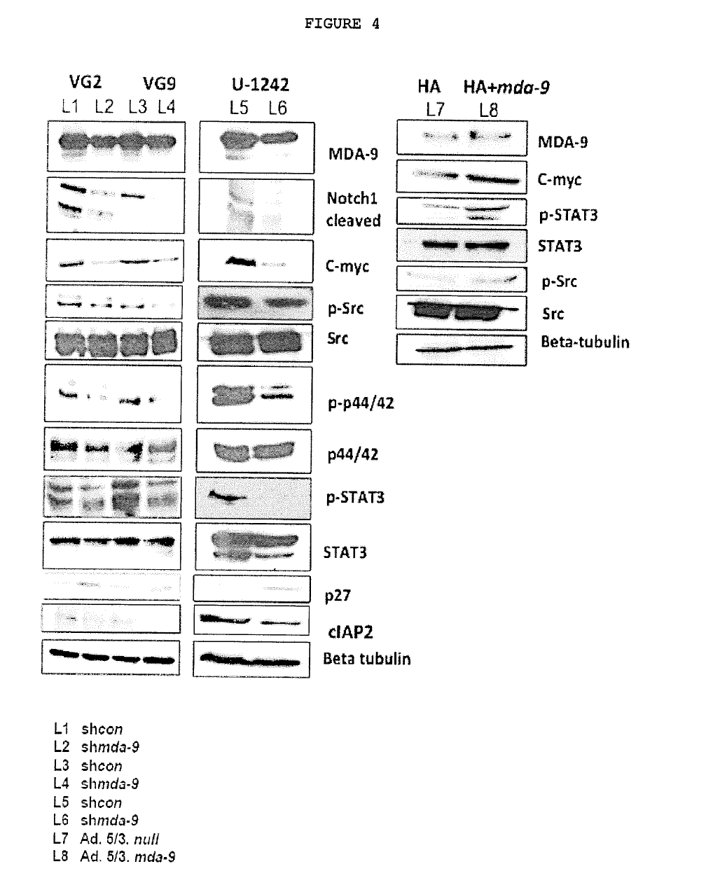

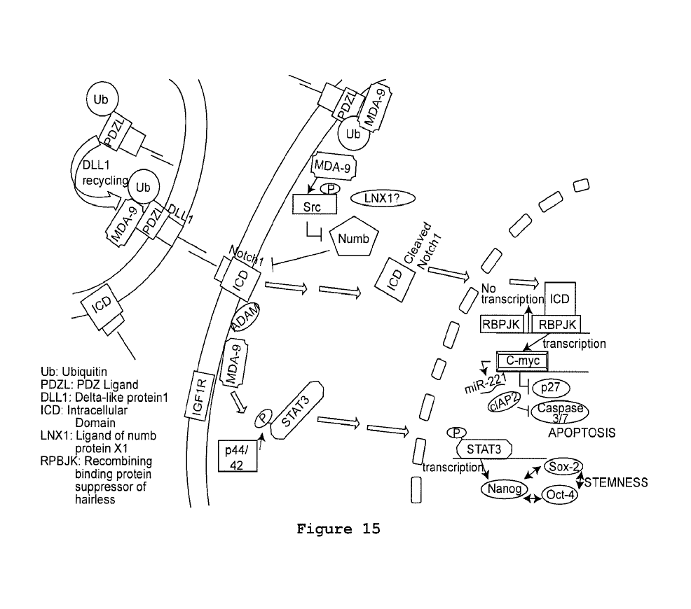

CSCs, also called cancer initiating cells, are considered defining elements in the carcinogenic process, hypothesized to represent critical constituents of invasion, angiogenesis, cancer cell resistance to therapy and escape of tumor cells from dormancy (tumor recurrence/relapse occurring after an initial therapeutic response) (40-42). MDA-9 is a diagnostic marker of tumor aggression and grade, and a positive association has been reported between MDA-9 expression and glioma stage (21). This Invention demonstrates a fundamental and central role of MDA-9/Syntenin as an upstream regulator of stemness and CSC survival in multiple human cancers, including GBM, and prostate and breast carcinomas. MDA-9 contributes to CSC cell-cell/cell-matrix adhesion, invasion, angiogenesis and metastasis. Stem cell-mediated cancer progression is a major clinical problem (5, 9, 17, 19) and is accentuated as a significant contributor to therapy-resistance and cancer relapse (43). mda-9 expression positively correlated with stemness as confirmed by a direct association between expression of mda-9 and stem cell markers and genes, in both patient samples and cell lines. Loss or gain of mda-9 expression led to a corresponding loss or gain of cell surface stem markers (FIGS. 1E, 1F and 1G; FIG. 2C; and Table 3) as well as recognized self-renewal/pluripotency genes including Nanog, Oct4, Sox2 and c-Myc (FIGS. 1A, 1B, 2E, 2F, 12B and 13B; FIG. 4; Table 2 and Table 3). mda-9 expression was also significantly higher in CSCs than NSCCs and both were dramatically elevated as compared to corresponding normal stem cells (FIG. 1E; FIGS. 2D and 2E). mda-9 also regulated STAT3 expression (FIG. 4), which is a key contributor to cellular transformation and tumor maintenance, including GBM (15). Activation of a STAT3-mediated transcriptional network correlates with mesenchymal GBM transformation and poor prognosis (34, 36, 41, 45). STAT3 also regulates cancer self-renewal by systematically regulating canonical stemness genes including Nanog, Sox2, Oct4 (16, 33, 34) and myc (46, 47). NANOG also acts as a master switch of the central stemness transcriptional network, as OCT4/SOX2 bind to the proximal region of the Nanog promoter stimulating Nanog expression (14). NANOG, SOX2 and OCT4, also reciprocally bind to their individual promoter's, thereby forming an interconnected auto-regulatory network to maintain cell pluripotency and self-renewal (14). The data reveal that mda-9 is a key regulator of this core stem cell regulatory system through regulation of STAT3 (FIG. 15).

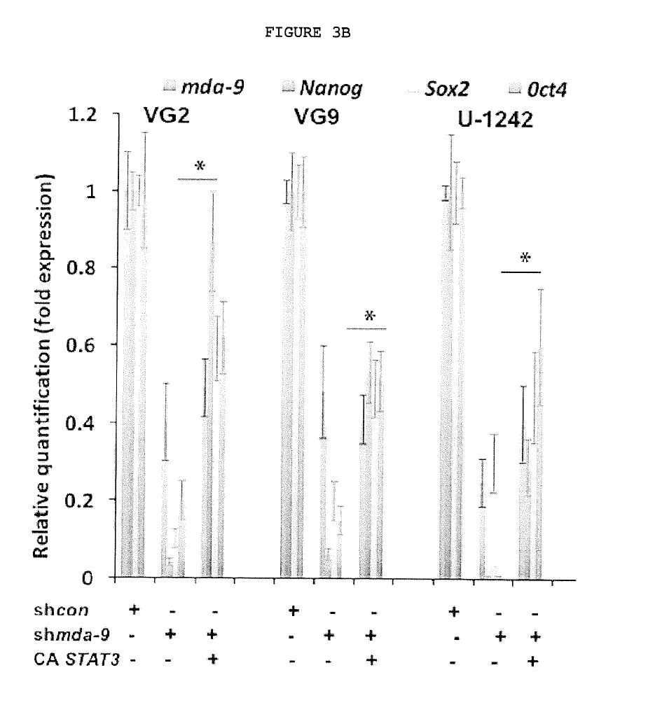

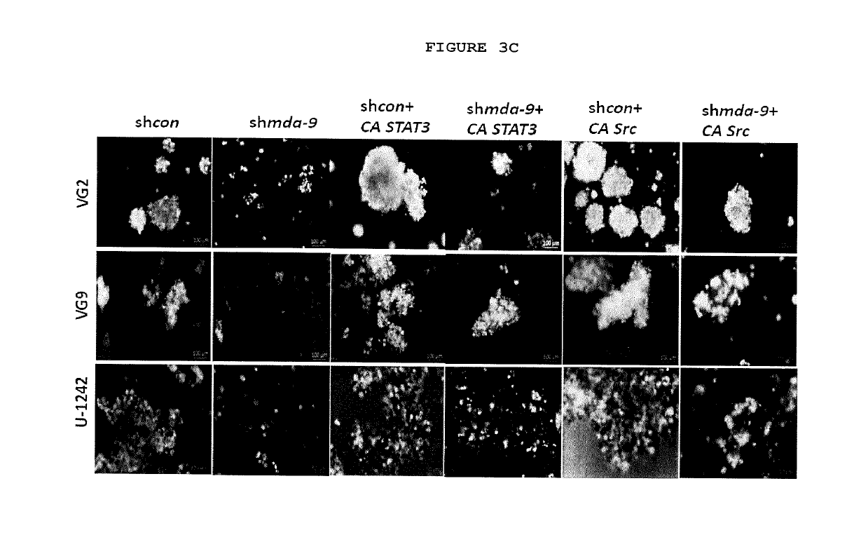

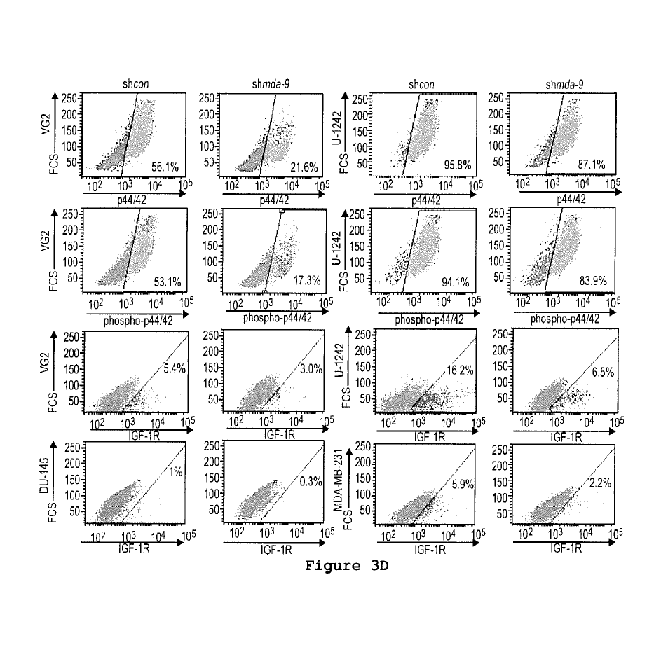

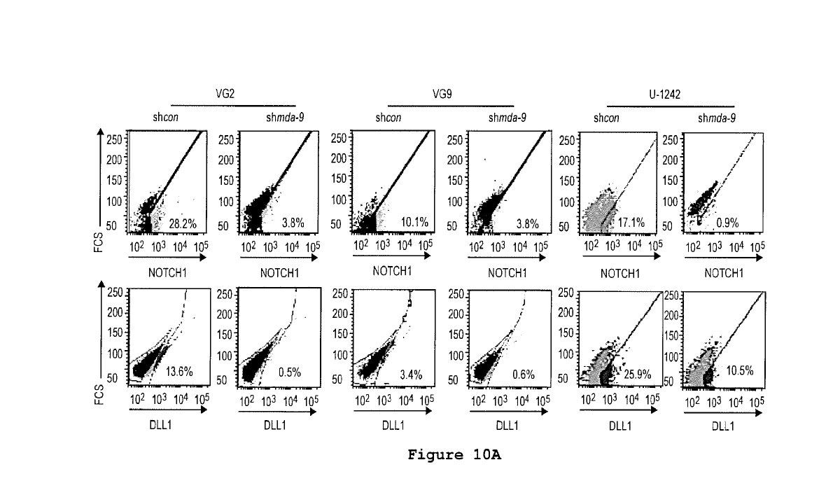

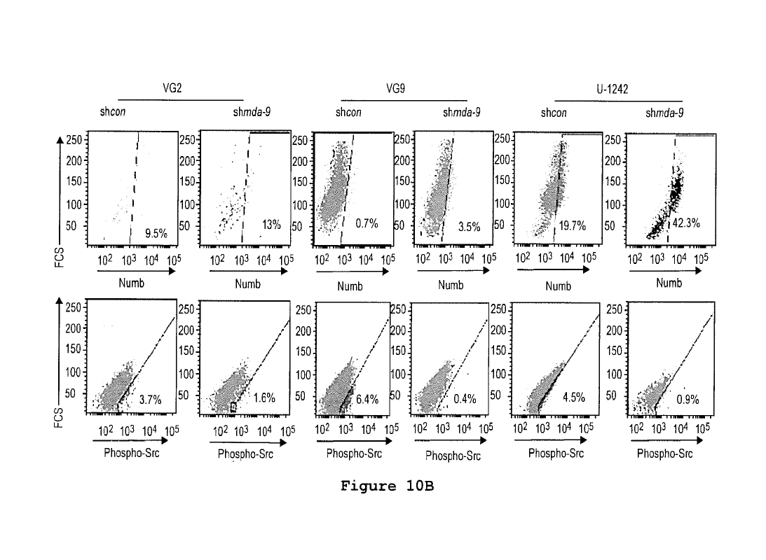

STAT3 can be regulated by SRC, IGF-1R, and p-44/42 (33-37, 43, 44, 47). Phosphorylated p-44/42 (T202/Y204) and SRC (T417, Y418) phosphorylate STAT3 at position Y705. The data indicates that MDA-9 regulates STAT3 by controlling IGF-1R (FIG. 3D), p-44/42 (FIG. 3D) and Src (FIG. 10B) signaling. MDA-9 also regulates the activity of FAK (10, 21, 22), RAF and RKIP (23, 24) and it ultimately controls the activation of p-44/42. MDA-9 physically interacts with c-SRC through its PDZ binding motifs and is essential for activation of SRC (21, 48). These data demonstrate that MDA-9 influences stemness on multiple molecular levels. The higher expression of MDA-9 in CSCs than in normal stem cells may indicate that CSCs are more dependent on mda-9 expression than their corresponding non-cancer stem cells. The potential “addiction” of CSCs to MDA-9 is an area of current investigation.

Another critical pathway in stem cell biology is the NOTCH pathway (15). NOTCH signaling plays an important role in development by regulating cell-fate determination, cell survival, and proliferation (16). Activation of NOTCH receptors occurs through binding with a number of distinct ligands (including delta-like 1/DLL1, jagged 1). Upon ligand binding, the intracellular NOTCH domain (ICD) is cleaved and translocates into the nucleus, where it regulates downstream target gene transcription. Aberrant NOTCH signaling promotes tumorigenesis (16). Recently, a role of the NOTCH signaling pathway in promoting self-renewal of both normal stem cells and CSCs has been demonstrated (16, 49). The data indicated that MDA-9 regulated NOTCH1 activity on two levels. NUMB, a NOTCH binding ubiquitin ligase regulated the expression of NOTCH1 in cells by degradation (FIG. 10B) (11, 50). In the presence of p-SRC, NUMB is phosphorylated and then degraded, preventing it from degrading NOTCH1 (51). In the absence of MDA-9, SRC is not activated to p-SRC (48) and this leads to higher expression of NUMB resulting in degradation and a decrease in the levels of total NOTCH1.

MDA-9 also controls NOTCH1 activity by regulating Notch1 activation through expression of DLL1, the ligand of the NOTCH1 receptor (FIG. 10A). The intracellular PDZ binding motif of DLL1 regulates DLL1 protein stability (52), DLL1 trafficking and signaling activity. DLL1 ubiquitination is not required for its internalization, but is necessary for its recycling back to the plasma membrane and efficient interaction with NOTCH1 (53). MDA-9 can regulate the expression of DLL1 on the cell surface by regulating the interaction between DLL1 and ubiquitin. An effect of MDA-9 on DLL1 has been reported in zebrafish stem cells (54). The c-terminal of MDA-9 binds to ubiquitin (55), and its PDZ domain may then bind to the PDZ binding motif of DLL1, and this interaction regulates the expression of DLL1 on the surface of CSCs. In the absence of MDA-9 this interaction is altered leading to decreased DLL1 surface expression. This further reduces the interaction of NOTCH1 with its ligand DLL1, leading to decreased activation of NOTCH1, reduced translocation of the intracellular domain (ICD) of Notch1 to the nucleus and decreased transcription of NOTCH1 target genes.

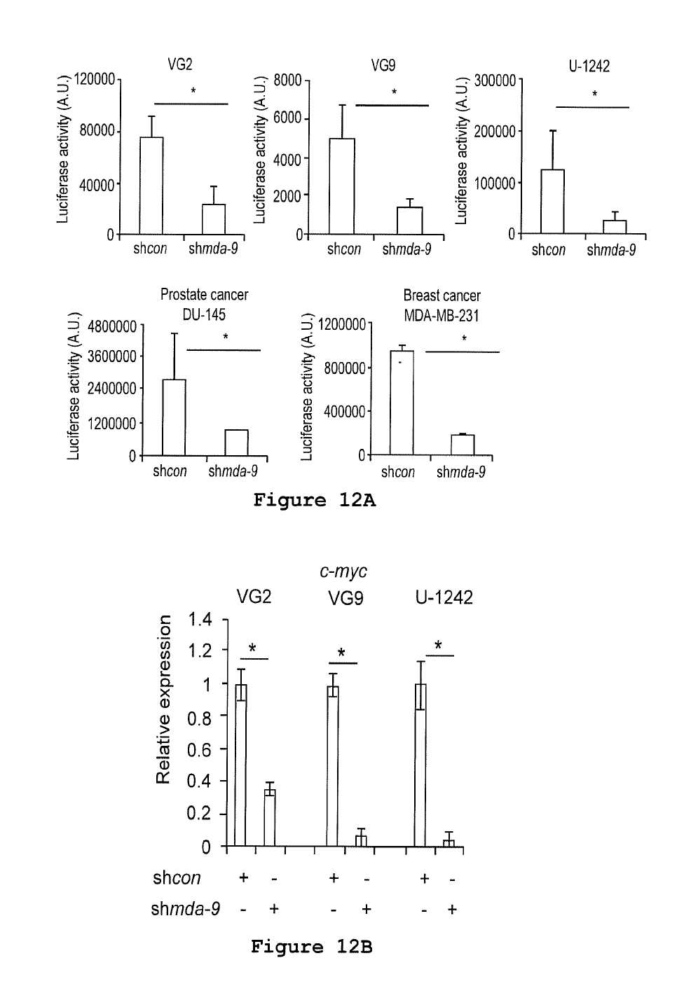

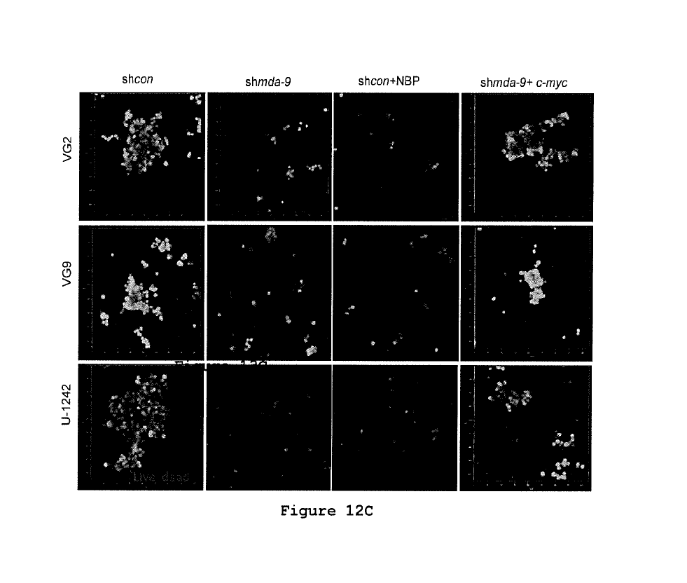

NOTCH1 directly regulates c-Myc expression (56). The ICD of NOTCH1 translocates to the nucleus and binds to the promoter of the transcription factor RBPJK, which regulates c-myc expression (57). The binding of NOTCH1 to the promoter region of RBPJK promotes expression of RBPJK, leading to expression of c-myc. In MDA-9 kd cells the ICD of NOCTH1 is unable to translocate to the nucleus, preventing transcription of RBPJK (FIG. 12A), thereby inhibiting elevated c-myc expression (FIG. 12B).

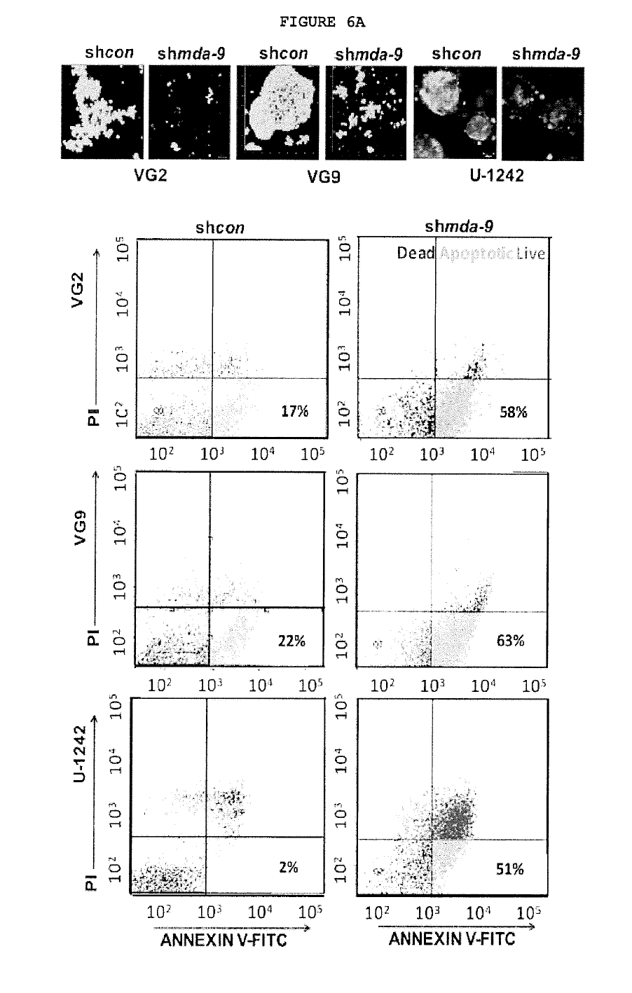

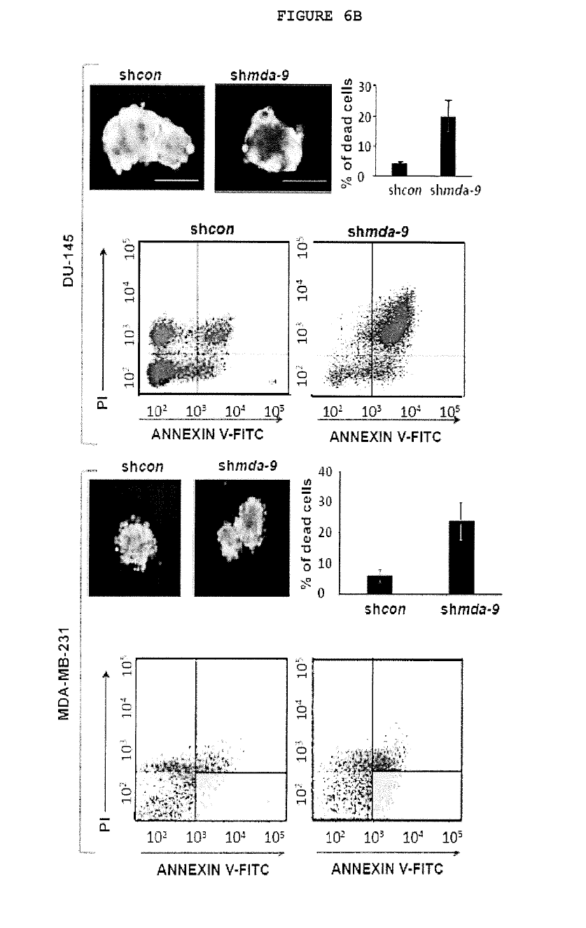

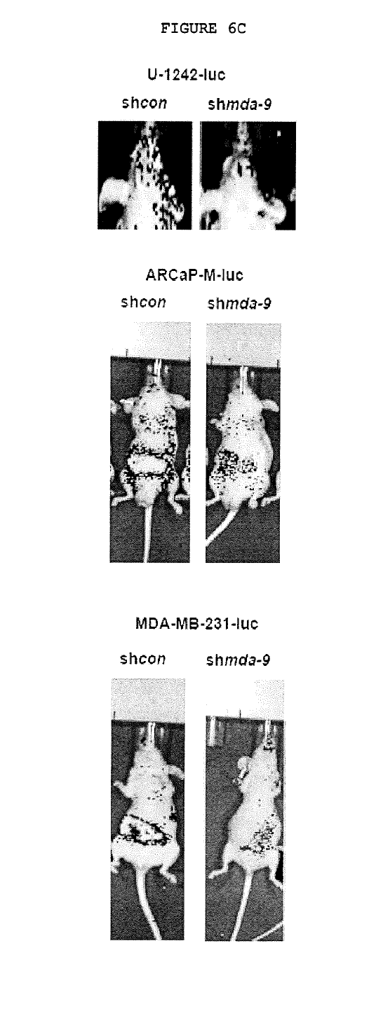

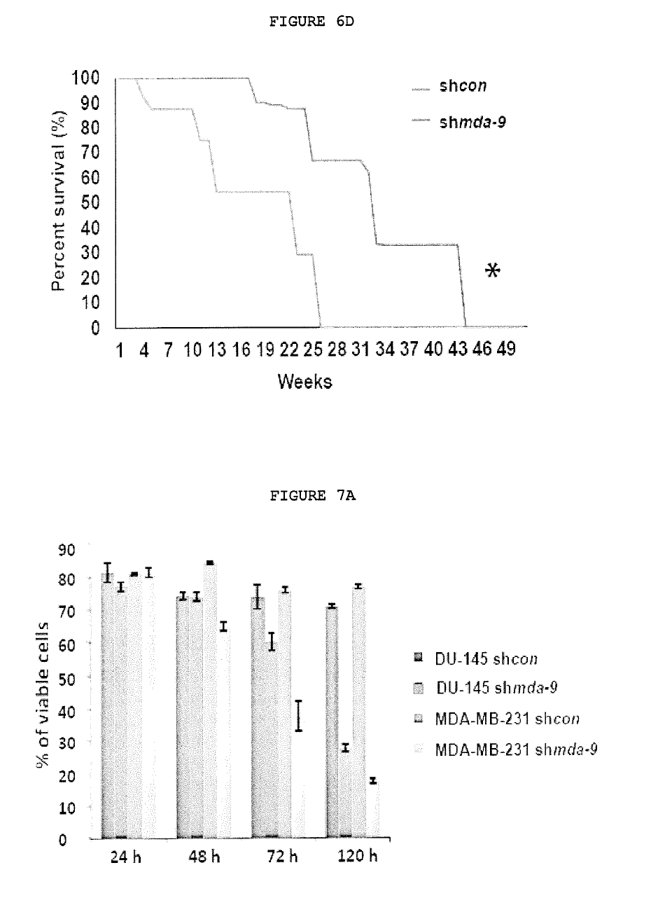

Elevated MYC proteins are associated with many cancers and correlate with cancer risk and poor patient survival (18, 58). Activation of MYC is linked to cellular growth, proliferation and metabolism. C-Myc controls the balance between stem cell self-renewal and differentiation in normal cells. In CSCs, C-Myc is essential for CSC initiation and maintenance (37, 38, 39). C-myc also controls the proliferation of cells by regulating cell cycle modulators including the cyclin-dependent kinase inhibitor, p27, which is a critical target of C-Myc (59). SRC has also been shown to negatively regulate p27 and elevated levels of p27 cause arrest of tumor growth and apoptosis (60). Additionally, p27 can suppress SOX-2 (61), which leads to apoptosis in stem cells (62). The data revealed that kd of mda-9 decreased SRC, Sox-2 and C-Myc activities, whereas p27/kip-1 expression was increased, culminating in apoptosis of CSCs (FIGS. 3B, 6A, 6B, 10B, 12B, 13B and 13C). Another anti-apoptotic molecule cIAP2, was also regulated by MDA-9 in CSCs (FIG. 4). IAP family members, XIAP, cIAP1, cIAP2, NAIP and survivin, are expressed at higher levels in CD133 positive than in CD133 negative GBM (63), and these anti-apoptotic proteins contribute to CSC survival under adverse conditions. Kd of MDA-9 expression decreased expression of cIAP2 (FIG. 4), which also participated in induction of apoptosis (FIG. 6A, 6B).

The current data suggests that MDA-9/syntenin is part of a complex, tightly regulated connectivity network that confers self-renewal, survival and tumor progressive properties to CSCs (64). Stemness, initially defined by the expression of cell surface markers and stem cell genes, is a property shared by normal stem cells and CSCs (65). MDA-9 appears to regulate stemness through similar pathways in both normal and CSCs. However, CSCs appear to be more dependent on (or “addicted” to) MDA-9, with significantly elevated expression (FIG. 1E), for maintenance and survival than normal stem cells. Forced elevated expression of MDA-9 in normal astrocytes, prostate and breast epithelial cells increased their invasiveness, self-renewal and the overall proportion of stem cells, but it did not render these cells tumorigenic. The regulation of stemness by MDA-9 is not exclusive to CSCs, but elevated expression enhances CSC survival, invasion, angiogenesis, metastasis and self-renewal. MDA-9 is capable of regulating multiple aspects of stem cell phenotypes simultaneously, validating a critical role in determining cancer stemness. mda-9 can regulate the central transcriptional network of stem regulating genes, additional pluripotency genes, and affects interrelated pathways crucial for stem cell survival (FIG. 15). Considering the pivotal role of MDA-9 in determining CSC aggressiveness and survival, directly targeting MDA-9 expression or its interaction with effector interacting proteins using genetic or pharmacological approaches may provide a unique opportunity to develop targeted therapies for this important component of cancer pathogenesis.

This invention will be better understood by reference to the examples which follow. However, one skilled in the art will readily appreciate that the examples provided are merely for illustrative purposes and are not meant to limit the scope of the invention which is defined by the claims following thereafter.

Throughout this application, it is to be noted that the transitional term “comprising”, which is synonymous with “including”, “containing” or “characterized by”, is inclusive or open-ended, and does not exclude additional, un-recited elements or method steps.

### Results

mda-9 Governs Stemness in Normal and Cancer Cells

A positive correlation between mda-9 expression, stemness and increasing tumor grade was evident in GBM (FIGS. 1A, 1B, and 1C). Forty-eight patient samples were assayed for c-myc, CD133, Nanog and mda-9 expression (FIGS. 1A and 1C). Data was normalized to 18S and beta tubulin expression and analyzed statistically by ANOVA. The results were statistically significant (R²=0.743, p<0.05), and a positive correlation was observed between mda-9 and myc (CI: 0.705), Nanog (CI: 0.574) and CD133 (CI: 0.505) expression (FIG. 1A). Correlation coefficients illustrate the relationship and intensity between variables, with values between −1 to 1, and CI is the confidence of the correlation, 1 indicating a 100% correlation. Based on these observations, the control and mda-9 knockdown (kd) (shmda-9) CSCs from a clinical GBM sample (VG2) were assayed by using a cancer stem cell array (Human Cancer Stem Cells RT2 Profiler PCR array, Qiagen/Sabiosciences) (FIG. 1B). Eighty-four genes were examined, and kd of mda-9 significantly affected a spectrum of pluripotency genes and the STAT3 pathway. The genes most affected by mda-9 kd in CSCs (downregulated a minimum of 4-fold by selecting the statistical boundary for Log₁₀ shmda-9 del del CT/Log₁₀ shcon del del CT as 4) were ALDH1A1, AXL, CD44, DDR1, DKK1, ID1, ITGB1, MYC, NANOG, OCT4/POU5F1, SOX2 and STAT3 (FIG. 1B). All of these genes, except for DKK1, promote stemness. Additionally, AXL is an important target for chemoresistance (32). An increase in mda-9 expression was also evident in cancer stem cells (CSCs)>non-stem cancer cells (NSCCs)>normal stem cells (SCs) (FIGS. 1E, 2D and 2E).

mda-9 mRNA levels were quantified in different stem and non-stem cell populations of glioblastomas, from both cell lines and clinical samples, as well as from prostate and breast cancer cell lines. In all samples, increased mda-9 expression was observed in stem vs. non-stem populations (Table 1). mda-9 expression in non-stem U-1242 cells, non-stem cancer cells (NSCC), was ˜35-fold greater than in primary adult human astrocyte (HA) stem cells (FIG. 1E, top right panel). Additionally, the expression of mda-9 in U-1242 CSCs was double that of U-1242 NSCCs (FIG. 1E, top right panel). Similarly, DU-145 CSCs expressed ˜40-fold more mda-9 than immortal normal human prostate epithelial (RWPE-1) stem cells (FIG. 2D). Since CSCs expressed higher levels of stemness genes than corresponding non-stem cells, the relationship between mda-9 expression and stemness in CSCs vs. NSCCs was examined. Elevated mda-9 expression directly correlated with stemness (Table 2), mda-9:Nanog (Pearson's correlation coefficient R=0.838, coefficient of determination R²=0.7034), mda-9:Sox2 (R=0.968, R²=0.937), mda-9:Oct4 (R=0.836, R²=0.698) and mda-9:c-Myc (R=0.954, R²=0.911).

TABLE 1
Expression of mda-9 in non-stem and CSCs of various tumor
lineages and from GBM clinical samples.

| | Non-stem cancer cell | Cancer stem cell |
|---|---|---|
| Cell lines | | |
| DU-145 | 1 ± 0.20 | 10.5 ± 0.10 |
| PC-3 | 1 ± 0.16 | 3.4 ± 0.25 |
| ARCaP-M | 1 ± 0.21 | 8.3 ± 0.07 |
| MDA-MB-231 | 1 ± 0.22 | 7.9 ± 0.38 |
| ZR-751 | 1 ± 0.10 | 6.9 ± 0.23 |
| C8161.9 | 1 ± 0.32 | 14.2 ± 0.04 |
| MeWo | 1 ± 0.11 | 12.1 ± 0.20 |
| U-1242 | 1 ± 0.04 | 2.9 ± 0.01 |
| U-87 MG | 1 ± 0.07 | 2.7 ± 0.04 |
| Clinical sample (GBM) | | |
| VG2 | 1 ± 0.03 | 5.6 ± 0.04 |
| VG9 | 1 ± 0.05 | 7.7 ± 0.20 |

TABLE 2
Expression of mda-9 and stemness genes in NSCCs,
CSCs from GBM, DU-145 and MDA-MB-231 cells.

| | sample | | | | | |
|---|---|---|---|---|---|---|
| | VG2 | | VG9 | | U-1242 | |
| Cell line | Non-stem | Glioma | Non-stem | Glioma | Non-stem | Glioma |
| GENES | glioma cell | stem cell | glioma cell | stem cell | glioma cell | stem cell |
| mda-9 | 1 ± 0.04 | 6.7 ± 1.20 | 1 ± 0.20 | 5.2 ± 0.44 | 1 ± 0.03 | 10.4 ± 0.12 |
| Stemness genes | | | | | | |
| Nanog | 1 ± 0.20 | 15.7 ± 0.46 | 1 ± 0.05 | 11.5 ± 0.79 | 1 ± 0.07 | 11.2 ± 2.20 |
| Sox2 | 1 ± 0.07 | 2.0 ± 0.70 | 1 ± 0.03 | 2.0 ± 0.82 | 1 ± 0.48 | 1.8 ± 0.08 |
| Oct4 | 1 ± 0.09 | 19.8 ± 2.70 | 1 ± 0.31 | 15.6 ± 1.54 | 1 ± 0.90 | 5.5 ± 0.25 |
| c-myc | 1 ± 0.42 | 9.1 ± 0.81 | 1 ± 0.02 | 8.7 ± 0.05 | 1 ± 0.10 | 10.3 ± 1.03 |
| Notch1 | 1 ± 0.06 | 4.1 ± 0.15 | 1 ± 0.10 | 3.5 ± 0.03 | 1 ± 0.61 | 3.7 ± 0.19 |

| | |
|---|---|
| DU-145 | MDA-MB-231 |

| Cell line | Non-stem | Cancer stem | Non-stem | Cancer stem |
|---|---|---|---|---|
| GENES | cancer cell | cell | cancer cell | cell |
| mda-9 | 1 ± 0.02 | 3.4 ± 0.05 | 1 ± 0.04 | 2.2 ± 0.10 |
| Stemness genes | | | | |
| Nanog | 1 ± 0.07 | 10.7 ± 0.03 | 1 ± 0.11 | 10.1 ± 0.40 |
| Sox-2 | 1 ± 0.04 | 2.9 ± 0.20 | 1 ± 0.06 | 2.4 ± 0.02 |
| Oct-4 | 1 ± 0.01 | 18 ± 0.07 | 1 ± 0.01 | 1.9 ± 0.03 |
| c-myc | 1 ± 0.06 | 2.3 ± 0.15 | 1 ± 0.05 | 2.6 ± 0.04 |

Forced MDA-9 overexpression in normal cells, led to a significant increase in spheroid size (FIG. 1E, top left panel; FIG. 2A), stem populations (FIG. 1E, bottom left panel; FIGS. 2A and 2B), self-renewal and pluripotency (FIGS. 1D, 1E, and 2F) as reflected by assessment of putative CSC and NSCC populations as well as changes in genes involved in self-renewal. No change in tumorigenicity was observed (data not shown). Overexpression of MDA-9 in NSCCs, significantly increased the stem population and expression of canonical stem regulatory genes (FIG. 1F-1G; 2C). Even though NSCC populations had elevated expression of mda-9, the CSC populations had significantly higher expression than the corresponding normal brain and normal prostate stem cells (FIGS. 1E and 2D). To further confirm that MDA-9 regulates stem regulatory genes mda-9 was suppressed by kd in GBM (cell line and clinical samples, n=5), and prostate and breast cancer cell lines. Silencing of mda-9 significantly decreased the recognized stem regulatory genes and markers (Table 3). Overall, Nanog was decreased by ˜33-, ˜25- and ˜11-fold, Oct4 by ˜7-, ˜12- and ˜2-fold, and Sox2 by ˜10-, ˜7- and ˜4-fold in the mda-9 kd GSCs from VG2, VG9, and U-1242, respectively. Silencing of mda-9 also resulted in significant loss of self-renewal (FIG. 1D) as defined by the self-renewal assays. While in the mda-9 kd for CSCs from DU-145, ARCaP-M and MDA-MB-231 cells, Nanog was decreased by 16.9±9.7-fold, Oct4 by 5.5±4.3-fold, and Sox2 by 6.7±3.1-fold, respectively. In total, these data support the hypothesis that mda-9 can regulate stemness in both normal stem cells and CSCs.

TABLE 3
Expression of mda-9 and stemness genes in control and shmda-9
GBM GSCs, and CSCs derived from DU-145 and MDA-MB-231 cells.

| | U-1242 | | VG2 | | VG9 | |
|---|---|---|---|---|---|---|
| GENES | shcon | shmda-9 | shcon | shmda-9 | shcon | shmda-9 |
| mda-9 | 1 ± 0.20 | 0.10 ± 0.01 | 1 ± 0.02 | 0.10 ± 0.01 | 1 ± 0.36 | 0.12 ± 0.01 |
| Stemness genes | | | | | | |
| Nanog | 1 ± 0.19 | 0.09 ± 0.01 | 1 ± 0.04 | 0.03 ± 0.10 | 1 ± 0.42 | 0.04 ± 0.02 |
| Sox2 | 1 ± 0.11 | 0.22 ± 0.06 | 1 ± 0.53 | 0.10 ± 0.03 | 1 ± 0.53 | 0.15 ± 0.05 |
| Oct4 | 1 ± 0.03 | 0.45 ± 0.03 | 1 ± 0.34 | 0.15 ± 0.03 | 1 ± 0.30 | 0.08 ± 0.02 |
| c-myc | 1 ± 0.41 | 0.11 ± 0.02 | 1 ± 0.19 | 0.09 ± 0.02 | 1 ± 0.25 | 0.06 ± 0.01 |

| | |
|---|---|
| DU-145 | MDA-MB-231 |

| GENES | shcon | shmda-9 | shcon | shmda-9 |
|---|---|---|---|---|
| mda-9 | 1 ± 0.07 | 0.20 ± 0.15 | 1 ± 0.06 | 0.10 ± 0.12 |
| Stemness genes | | | | |
| Nanog | 1 ± 0.02 | 0.14 ± 0.04 | 1 ± 0.02 | 0.13 ± 0.02 |
| Sox2 | 1 ± 0.06 | 0.32 ± 0.01 | 1 ± 0.08 | 0.10 ± 0.05 |
| Oct4 | 1 ± 0.11 | 0.55 ± 0.04 | 1 ± 0.05 | 0.24 ± 0.01 |
| c-myc | 1 ± 0.01 | 0.17 ± 0.03 | 1 ± 0.02 | 0.10 ± 0.03 |

mda-9 Regulates Stemness Through STAT3

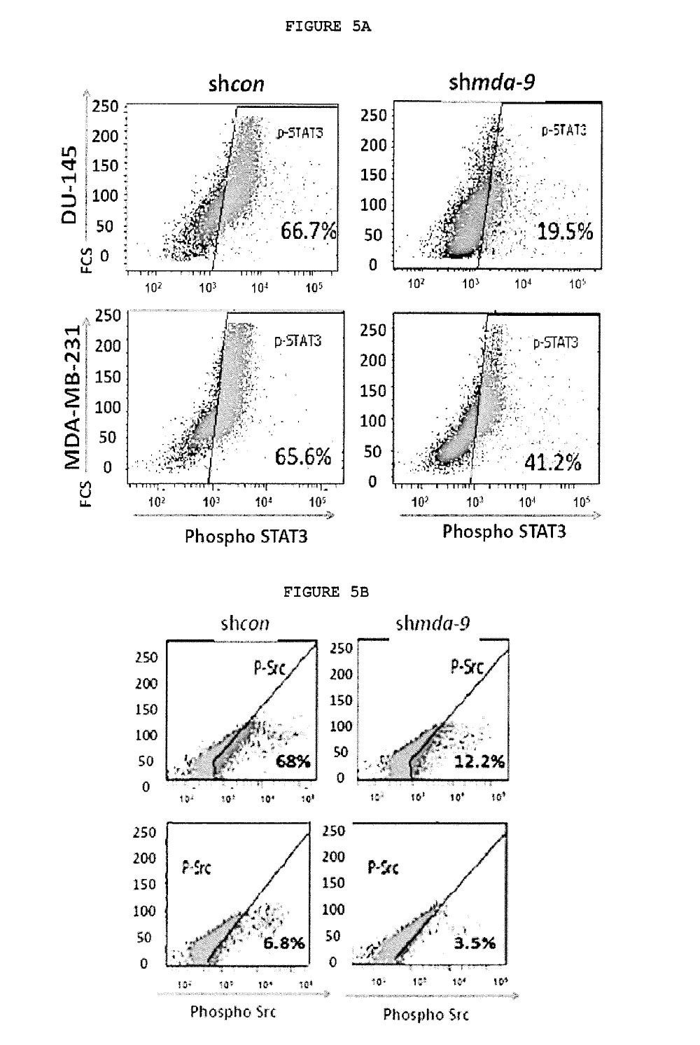

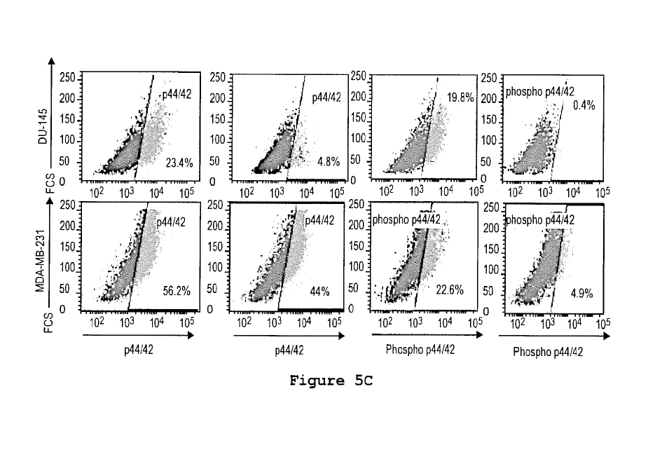

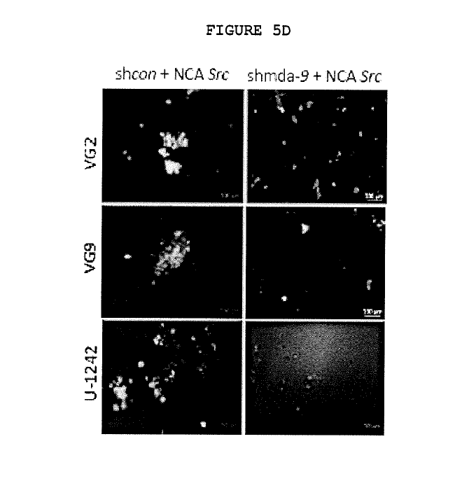

STAT3 is indispensable for the regulation of self-renewal in human stem cells including GSCs (17, 33, 34). Considering the seminal role of STAT3 as a regulator of stemness (17), this invention investigated the effect of mda-9 expression on STAT3. Kd of mda-9 significantly decreased the expression of p-STAT3 (FIG. 3A, FIG. 4 and FIG. 5A). p-STAT3 expression was decreased ˜2-4-fold overall in shmda-9 cells (32.0±6.3% decrease in VG2; 12.1±3.9% in VG9; 40.0±6.0% in U-1242; 39.2±6.2% in DU-145; and 21.2±5.4% in MDA-MB-231). To confirm further the hypothesis, mda-9 was overexpressed in primary normal cells and it was found that mda-9 overexpression lead to a significant increase in p-STAT3 (FIG. 4). The effects of mda-9 silencing were significantly attenuated by overexpressing a constitutively active STAT3 (A662C/N664C; CA STAT3) (FIG. 3C). Since active SRC positively regulates STAT3 (35), the constitutively active SRC (Y529F; CA Src) was overexpressed and a significant recovery of STAT3 function in the shmda-9 cells was once again observed (FIG. 3C). However, overexpression of a non-constitutively-active Src (NCA Src) failed to enhance STAT3 rescue function in the shmda-9 CSCs (FIG. 5D). As STAT3 is also regulated by p44/42 and IGF-1R (32, 36, 37), the expression of these proteins in control and shmda-9 CSCs were also measured. It was observed that some decrease in p44/42, a significant decrease in phospho-p44/42 (31.4±6.2% decrease in VG2; 62.0±7.9% decrease in VG9; 9.5±2.7% decrease in U-1242; 15.0±4.4% decrease in DU-145; 12.5±5.9% decrease in MDA-MB-231) (FIG. 3D; FIGS. 4 and 5C), and IGF-1R (˜2 to ˜3-folds) in the shmda-9 cells (FIG. 3D).

### MDA-9 Regulates Stem Cell Survival, Growth, Tumorigenicity and Metastasis

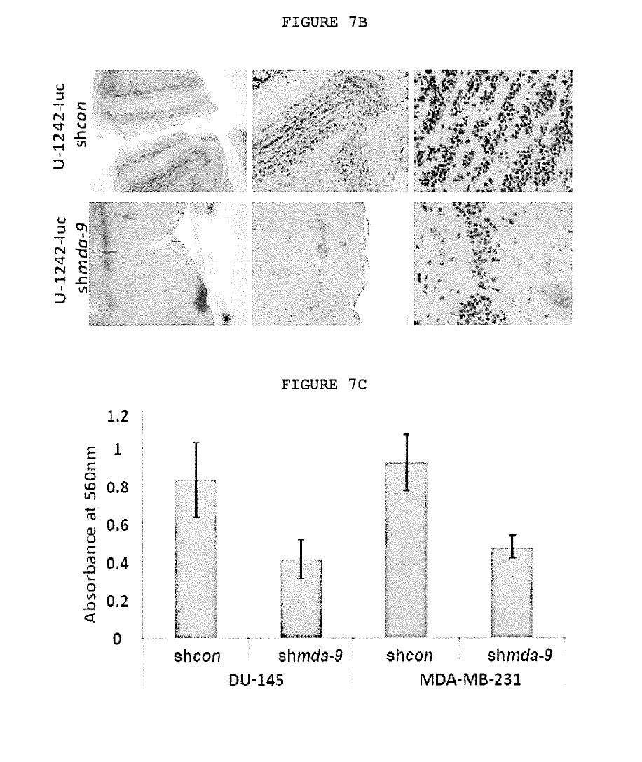

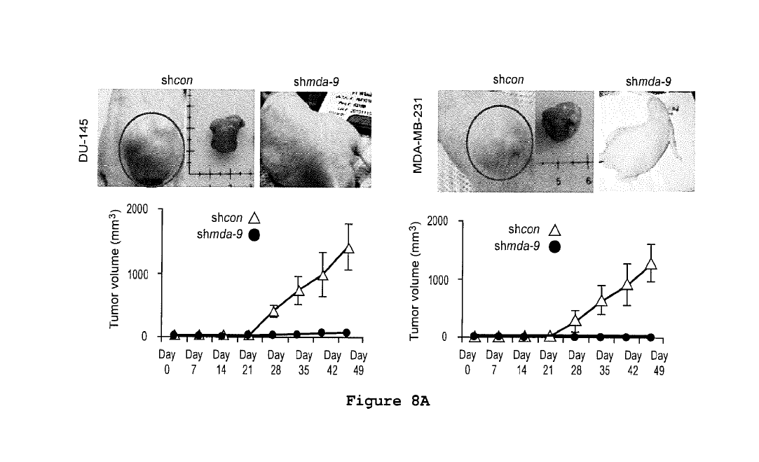

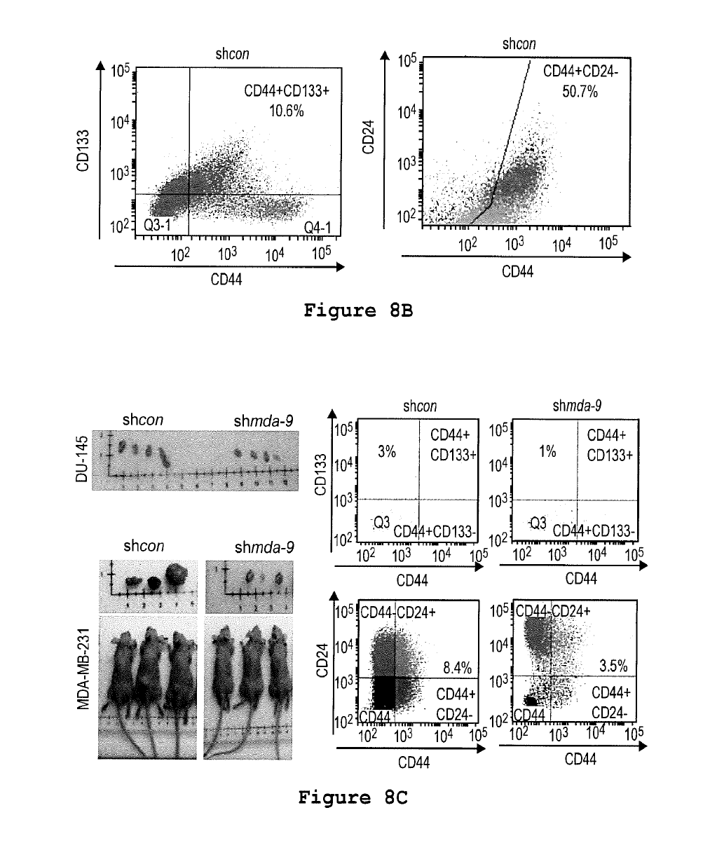

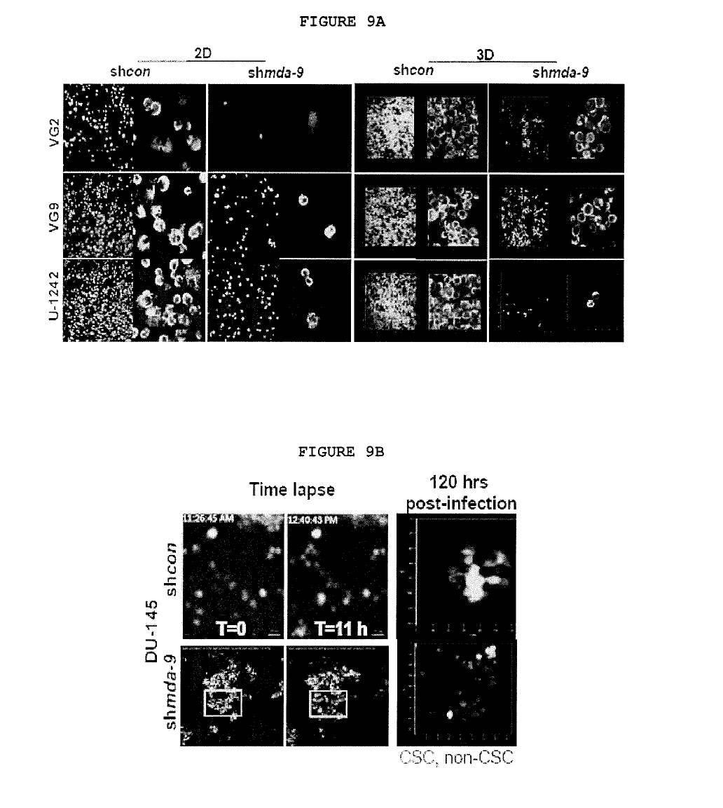

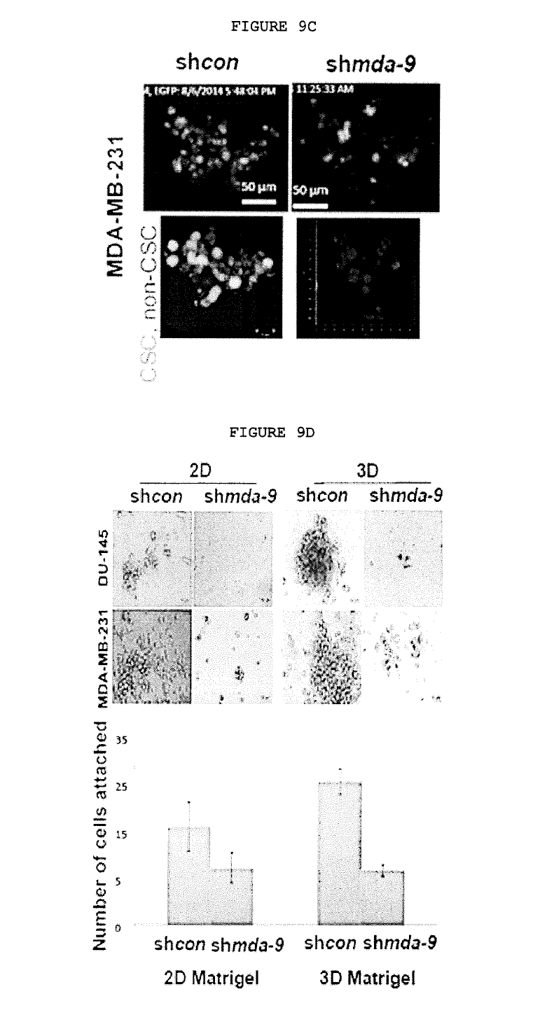

MDA-9 kd led to increased apoptotic cell death in CSCs (FIGS. 6A and 6B). Overall, the population of apoptotic cells in shmda-9 CSCs was 57.3±3.7% after 72 hr, which was ˜5-fold of that observed in shcon cells. The population of apoptotic cells in shmda-9 GSCs was 38±3.3%, 36±5.1% and 45±4.9% (in VG2, VG9 and U-1242, respectively) after 72 hours, which was ˜5-fold of that observed in shcon GSCs. Dead cells increased to 77.5±7.3% after 120 hr (FIG. 7A). MDA-9 suppression also resulted in a significant loss in CSC tumorigenicity and metastasis in vivo (FIG. 6C; FIG. 7B; FIG. 8; p<0.05). The control mice showed spongioblastic tumors with rhythmic palisades, a constant feature of aggressive high grade glioblastoma. Tumors in mice injected with shmda-9 GSCs were extremely small, and did not display the distinguishing aggressive spongioblastic pattern (FIG. 7B). In addition to causing decreased tumor growth, silencing mda-9 also significantly decreased the number of CSCs in vivo (FIG. 8C). mda-9 kd also significantly inhibited 2D- and 3D-stem cell attachment, spreading, anchorage-dependent and anchorage-independent growth (FIGS. 7C and 9).

### MDA-9 Regulates Stem Survival Through NOTCH1 Signaling

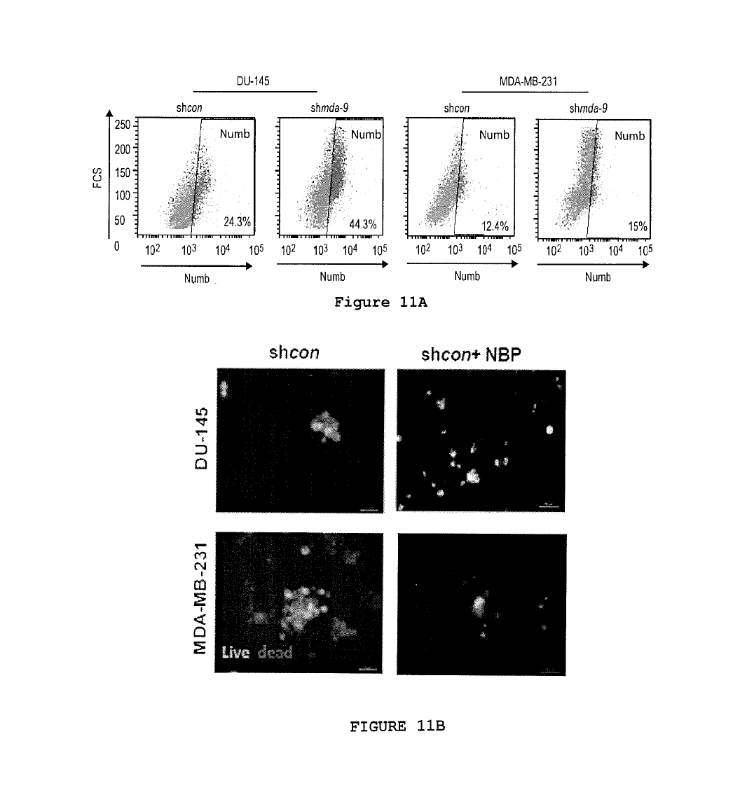

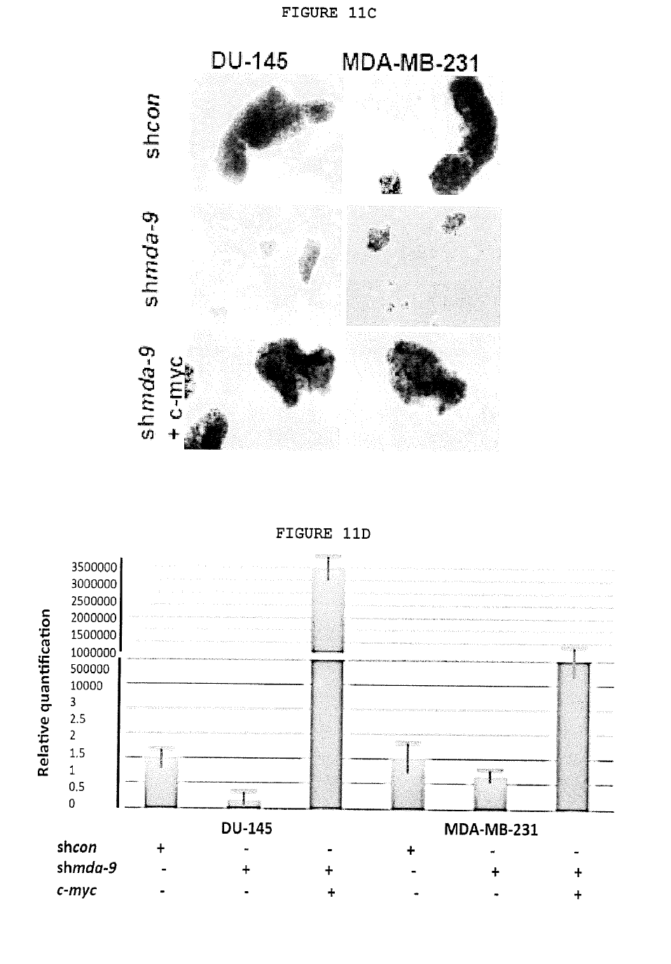

NOTCH1 expression was decreased ˜2.7-19.2-fold following kd of mda-9 in CSCs (FIGS. 4 and 10A). Decreased mda-9 expression led to NOTCH1 degradation through increased expression of NUMB (˜1.5-5-fold increase) and decreased p-SRC expression (˜2.1-16-fold decrease in relative expression) (FIG. 10B; FIG. 11A). In VG2, VG9 and U-1242 GSCs, decreased mda-9 expression led to NOTCH1 degradation through increased expression of NUMB (1.3±0.7, 4.8±0.4, 2±0.5-fold increase, respectively) and decreased p-SRC expression (2±0.9, 15.8±1.2, 5.5±0.4-fold decrease in relative expression, respectively) in VG2, VG9, and U-1242 GSCs (FIG. 10B). mda-9 kd also caused a loss of NOTCH1 activation (˜3-15.3-fold reduction of DLL1 in the test) (FIG. 10A). Blocking NOTCH1 recapitulated the phenotype observed with mda-9 kd (FIG. 11B; FIG. 12C). The decreased activity of NOTCH1 in shmda-9 cells lead to a significant decrease in RBPJK expression (FIG. 12A). The effect of mda-9 kd was rescued by expressing a constitutively active SRC (CA Src), but not with a non-constitutively-active SRC (NCA Src) (FIG. 5). Additionally, partial recovery from mda-9 kd occurred with addition of a DLL1 peptide (Data not shown).

MDA-9 Regulates Stemness and Stem Cell Survival Through c-Myc

Considering C-Myc's influential role in stem cell renewal, maintenance, and survival (38, 39), we investigated the role of MDA-9-mediated regulation of C-myc in HA SCs and GSCs. Suppression of mda-9 by kd and enhanced expression of mda-9 with an expression vector lead to a significant decrease (9.4±0.83-fold) or gain of c-myc (3.3±0.27-fold) expression, respectively. In VG2, VG9 and U-1242 GSCs, suppression of mda-9 by kd and enhanced expression of mda-9 with an expression vector lead to a significant decrease (˜3-, ˜2- and ˜5-fold protein, and ˜3-, ˜10- and ˜12-fold mRNA in VG2, VG9 and U-1242 GSCs, respectively) or gain of C-Myc (˜3-fold protein in HA) expression, respectively (FIG. 4; FIG. 12B; Table 3). The change in C-Myc was observed at both an RNA and protein level (FIG. 4; FIG. 12B; Table 3). This loss of c-myc expression phenotype in shmda-9 CSCs was reversed by c-myc overexpression (FIG. 11; FIG. 12C). mda-9 regulation of c-myc occurred though RBPJK transcription, which is possibly regulated by NOTCH1 cleavage/activation (FIG. 12) via interaction with its ligand, DLL1 (FIG. 10A). These findings support the concept that MDA-9 plays a critical role in the regulation of C-Myc in GSCs, which is a major contributor of glioma stemness and GSC survival (38) via the activation of NOTCH1 and RBPJK.

MDA-9 Regulates CSC Survival Through p27/Kip-1 and cIAP2

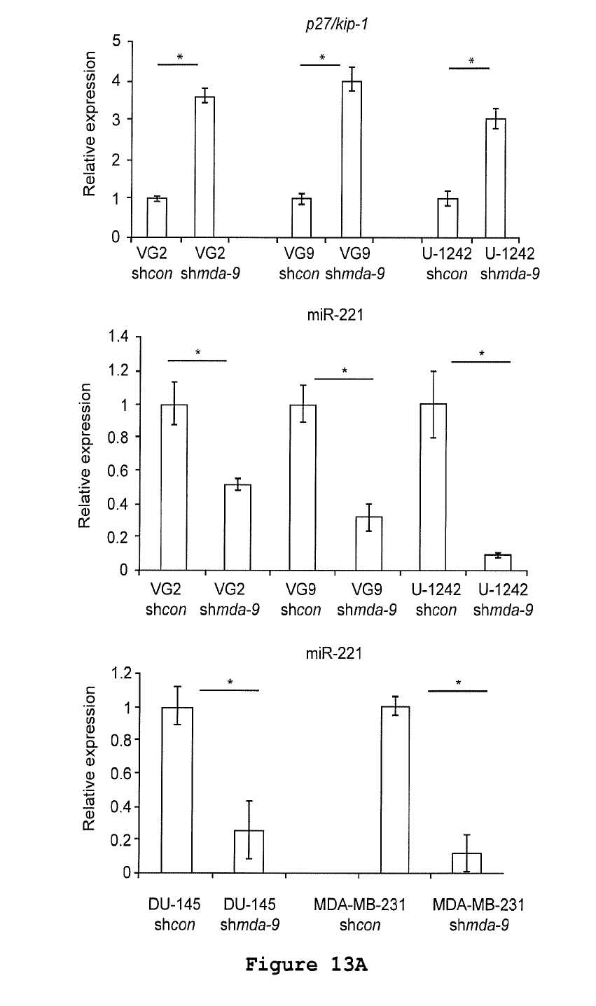

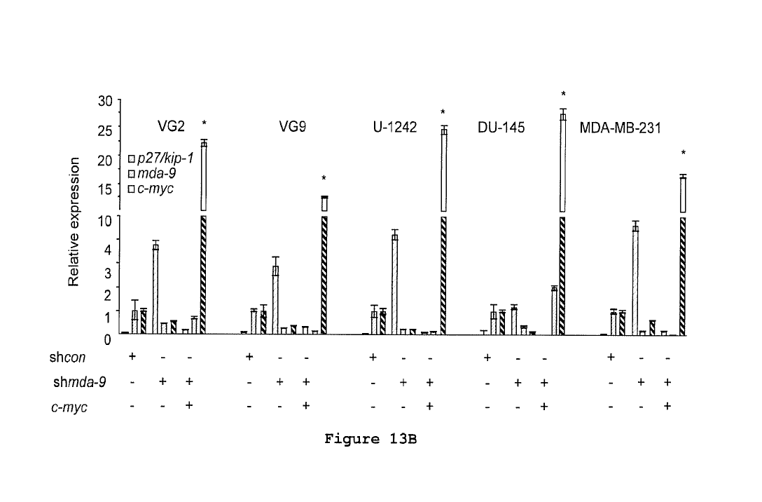

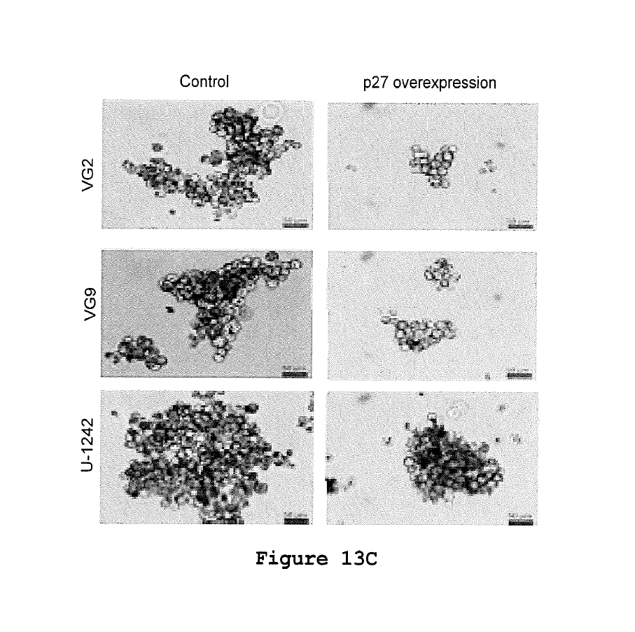

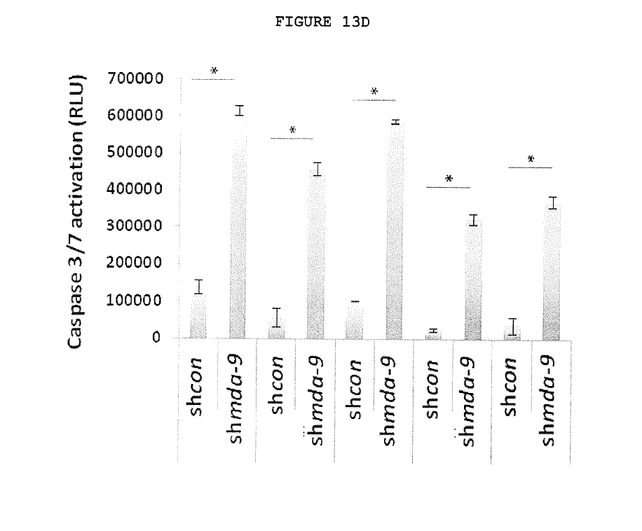

Kd of mda-9 led to increased expression of p27 in GBM, prostate and breast CSCs at both an RNA and protein level (FIG. 4; FIGS. 13A and 13B). The increased expression of p27 that culminated in cell death could be prevented by forced expression of c-myc, indicating that CSC survival is dependent on c-myc and p27 expression (FIG. 13B). In the shmda-9 CSCs, expression of miR-221 was also significantly decreased (FIG. 13A). These findings demonstrate that p27 is regulated by mda-9 through c-myc and miR-221. kd caused decreased cIAP2 expression (FIG. 4) and this combined with increased expression of p27 in shmda-9 CSCs may amplify CSC death. To verify p27's involvement in CSC survival, p27 in CSCs was overexpressed and a loss of sphere integrity and viability was observed, in both patient-derived GBM and the U-1242 GBM cell line (FIG. 13C). It was also observed that cell death in shmda-9 CSCs was mediated by Caspase activation (FIG. 13D).

### MDA-9 Regulates CSC Angiogenesis

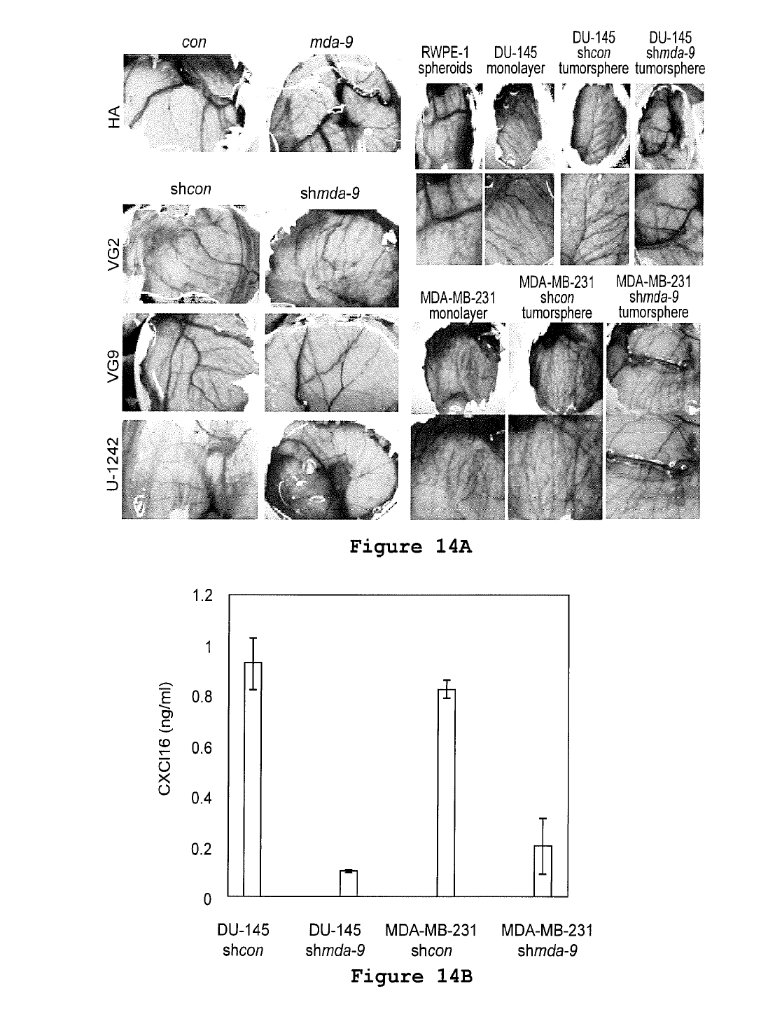

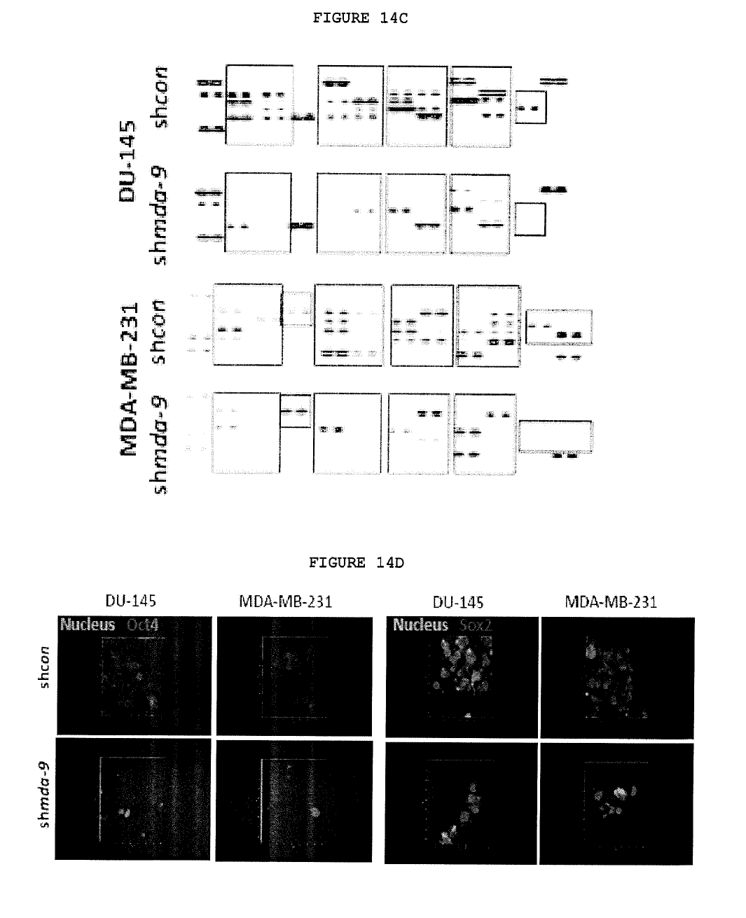

CSCs play a prominent role in tumor progression and to achieve this activity both invasive and angiogenic abilities are crucial (2). Prior studies indicate a critical role of mda-9 in cancer cell angiogenesis and invasion (10, 20-23). Overexpression and kd of mda-9 in stem cells led to a gain and loss of invasive and angiogenic activity, respectively (FIGS. 7, 14A, 14B, 14C and 14D; Table 4). Several pivotal molecules involved in angiogenesis, including angiogenin, CXC116, and IGFBP2, were decreased following kd of mda-9 in shmda-9 CSCs from DU-145 and MDA-MB-231 cells. Measurement of CXC116 levels by ELISA confirmed that mda-9 regulated angiogenesis in CSCs (FIG. 14B).

TABLE 4
Angiogenic protein array analysis of conditioned media
from control and mda-9 kd CSCs.

| | | | Effect of |
|---|---|---|---|
| | | | mda-9 kd on |
| Coordinate | Protein | Cell line | regulation |
| A1, A2 | Reference Spots | DU-145 | NA |
| | | MDA-MB-231 | NA |
| A5, A6 | Activin A | DU-145 | |
| | | MDA-MB-231 | Downregulated |
| A7, A8 | ADAMTS-1 | DU-145 | |
| | | MDA-MB-231 | |
| A9, A10 | Angiogenin | DU-145 | Downregulated |
| | | MDA-MB-231 | Downregulated |
| A11, A12 | Angiopoietin-1 | DU-145 | Downregulated |
| | | MDA-MB-231 | Downregulated |
| A13, A14 | Angiopoietin-2 | DU-145 | |
| | | MDA-MB-231 | Downregulated |
| A15, A16 | Angiostatin/Plasminogen | DU-145 | |
| | | MDA-MB-231 | |
| A17, A18 | Amphiregulin | DU-145 | Downregulated |
| | | MDA-MB-231 | |
| A19, A20 | Artemin | DU-145 | |
| | | MDA-MB-231 | |
| A23, A24 | Reference Spots | DU-145 | NA |
| | | MDA-MB-231 | NA |
| B1, B2 | Coagulation Factor III | DU-145 | Downregulated |
| | | MDA-MB-231 | Downregulated |
| B3, B4 | CXCL16 | DU-145 | Downregulated |
| | | MDA-MB-231 | Downregulated |
| B5, B6 | DPPIV | DU-145 | Downregulated |
| | | MDA-MB-231 | Downregulated |
| B7, B8 | EGF | DU-145 | |
| | | MDA-MB-231 | |
| B9, B10 | EG-VEGF | DU-145 | |
| | | MDA-MB-231 | |
| B11, B12 | Endoglin | DU-145 | |
| | | MDA-MB-231 | |
| B13, B14 | Endostatin/Collagen | DU-145 | Downregulated |
| | XVIII | MDA-MB-231 | Downregulated |
| B15, B16 | Endothelin-1 | DU-145 | Downregulated |
| | | MDA-MB-231 | Downregulated |
| B17, B18 | FGF acidic | DU-145 | |
| | | MDA-MB-231 | Downregulated |
| B19, B20 | FGF basic | DU-145 | Downregulated |
| | | MDA-MB-231 | Downregulated |
| B21, 23 | FGF-4 | DU-145 | |
| | | MDA-MB-231 | |
| B23, B24 | FGF-7 | DU-145 | |
| | | MDA-MB-231 | Downregulated |
| C1, C2 | GDNF | DU-145 | |
| | | MDA-MB-231 | |
| C3, C4 | GM-CSF | DU-145 | Downregulated |
| | | MDA-MB-231 | Downregulated |
| C5, C6 | HB-EGF | DU-145 | |
| | | MDA-MB-231 | Downregulated |
| C7, C8 | HGF | DU-145 | |
| | | MDA-MB-231 | |
| C9, C10 | IGFBP-1 | DU-145 | Downregulated |
| | | MDA-MB-231 | |
| C11, C12 | IGFBP-2 | DU-145 | Downregulated |
| | | MDA-MB-231 | Downregulated |
| C13, C14 | IGFBP-3 | DU-145 | |
| | | MDA-MB-231 | Downregulated |
| C15, C16 | IL-1β | DU-145 | |
| | | MDA-MB-231 | Downregulated |
| C17, C18 | IL-8 | DU-145 | Downregulated |
| | | MDA-MB-231 | Downregulated |
| C19, C20 | LAP (TGF-β1) | DU-145 | Downregulated |
| | | MDA-MB-231 | Downregulated |
| C21, C23 | Leptin | DU-145 | |
| | | MDA-MB-231 | |
| C23, C24 | MCP-1 | DU-145 | |
| | | MDA-MB-231 | |
| D1, D2 | MIP-1α | DU-145 | |
| | | MDA-MB-231 | |
| D3, D4 | MMP-8 | DU-145 | Downregulated |
| | | MDA-MB-231 | |
| D5, D6 | MMP-9 | DU-145 | Downregulated |
| | | MDA-MB-231 | Downregulated |
| D7, D8 | NRG1-β1 | DU-145 | |
| | | MDA-MB-231 | |
| D9, D10 | Pentraxin 3 (PTX3) | DU-145 | Downregulated |
| | | MDA-MB-231 | Downregulated |
| D11, D12 | PD-ECGF | DU-145 | Downregulated |
| | | MDA-MB-231 | Downregulated |
| D13, D14 | PDGF-AA | DU-145 | Downregulated |
| | | MDA-MB-231 | Downregulated |
| D15, D16 | PDGF-AB/PDGF-BB | DU-145 | Downregulated |
| | | MDA-MB-231 | Downregulated |
| D17, B18 | Persephin | DU-145 | |
| | | MDA-MB-231 | Downregulated |
| D19, D20 | Platelet Factor 4 (PF4) | DU-145 | Downregulated |
| | | MDA-MB-231 | Downregulated |
| D21, D22 | PlGF | DU-145 | Downregulated |
| | | MDA-MB-231 | Downregulated |
| D23, D24 | Prolactin | DU-145 | |
| | | MDA-MB-231 | |
| E1, E2 | Serpin B5 | DU-145 | Downregulated |
| | | MDA-MB-231 | |
| E3, E4 | Serpin E1 | DU-145 | |
| | | MDA-MB-231 | Downregulated |
| E5, E6 | Serpin F1 | DU-145 | Downregulated |
| | | MDA-MB-231 | Downregulated |
| E7, E8 | TIMP-1 | DU-145 | |
| | | MDA-MB-231 | Downregulated |
| E9, E10 | TIMP-4 | DU-145 | Downregulated |
| | | MDA-MB-231 | Downregulated |
| E11, E12 | Thrombospondin-1 | DU-145 | Downregulated |
| | | MDA-MB-231 | Downregulated |
| E13, E14 | Thrombospondin-2 | DU-145 | |
| | | MDA-MB-231 | |
| E15, E16 | uPA | DU-145 | |
| | | MDA-MB-231 | Downregulated |
| E17, E18 | Vasohibin | DU-145 | |
| | | MDA-MB-231 | |
| E19, E20 | VEGF | DU-145 | |
| | | MDA-MB-231 | Downregulated |
| E21, E22 | VEGF-C | DU-145 | Downregulated |
| | | MDA-MB-231 | Downregulated |
| F1, F2 | Reference Spots | DU-145 | NA |
| | | MDA-MB-231 | NA |
| F23, F24 | Negative Control | DU-145 | NA |
| | | MDA-MB-231 | NA |

### Materials and Methods

### Cell Line and Tissue Samples

RWPE-1 normal prostate epithelial cells, DU-145 prostate and MDA-MB-231 breast cancer cells were purchased from the American Type Culture Collection. The human glioma cell line U-1242-luc-GFP cells were kindly provided by Dr. Kristofer Valerie (VCU). U-1242/luc-GFP, DU-145 and MDA-MB-231 cells were cultured in DMEM medium supplemented with 10% fetal bovine serum and antibiotics. Isolated NSCCs, based on lack of expression of CD133 and CD44, were cultured similarly in monolayer culture. Normal human astrocytes (HA) were obtained from Clonetics, USA and grown in Clonetics EBM (Endothelial Cell Basal Media, No. CC-2131) supplemented with hydrocortisone (1 μg/ml), hEGF (20 ng/ml), insulin (25 μg/ml), progesterone (25 ng/ml), transferrin (50 μg/ml), and 5% fetal bovine serum. The cumulative culture length of the cells was less than 6 months after resuscitation. Early passage cells were used for all experiments and they were not reauthenticated. All the cell lines were frequently tested for mycoplasma contamination using a mycoplasma detection kit from Sigma. Specimens of human primary normal and malignant brain tumors (n=50) were collected from subjects who underwent surgical removal of their brain tumors. All subjects were informed of the nature and requirements of the study and provided written consent to donate their tissues for research purposes. Informed consent was obtained according to Origene and the research proposals approved by the Institutional Review Board at the VCU TDAAC.

### Isolation and Culture of Human GBM, Prostate and Breast CSCs and NSCCs

Human GBM CSCs and NSCCs were isolated from GBM tissue from surgical specimens and from established U-1242/luc-GFP GBM cells. Glioblastoma tissue samples were dissociated with Trypsin (Invitrogen), Hyaluronidase (Sigma), Collagenase (Sigma), and DNase I (Sigma) mixture. Enzyme reaction was stopped by Trypsin inhibitor (Sigma), followed by washing in PBS. Digested samples were filtered with 70 μm nylon cell strainer (BD) and resuspended in stem cell medium comprised of DMEM/F-12 50:50 containing K27 supplements, glutamine 2 μmol, (Invitrogen), basic fibroblast and epidermal growth factors (PeproTech, 20 ng/mL each) for continuous culturing (26). Floating neurospheres were amplified and stored for further experiments. All primary cells were cultured as suspended spheres in uncoated T25 or T75 culture dishes (BD) and analyzed prior to 5 passages. All primary tumor cells were authenticated by IDEXX Bioresearch (Columbia, Mo.). Neurospheres were disassociated with Accutase (Invitrogen) and then labeled with CD44 and CD133 antibody (Miltenyi Biotec). Stained cells were sorted through a BD Aria II sorting station. Antibody negative and positive cell populations were counted and collected for further culturing. The glioblastoma CSCs were cultured in ultra-low attachment plates and flasks (Corning) in the media specified above. CSCs were also isolated from DU-145, ARCaP-M-luc prostate carcinoma cells and MDA-MB-231 breast carcinoma cells. Prostate cancer cells were grown in ultra-low attachment flasks and then stained with CD44 and CD133. MDA-MB-231 cells were similarly stained with CD44 and CD24 (Miltenyi Biotec). Stained cells were sorted through a BD Aria II sorting station. CSC and NSCC populations were counted and collected for further culturing. The CSCs were cultured in ultra-low attachment plates and flasks with Essential 8 medium (Invitrogen), unless indicated. Isolated NSCCs were cultured in monolayer with complete DMEM medium. Xenografted human CSCs were isolated from mice and analyzed for cell surface and intracellular proteins by FACS. Informed consent was obtained according to the research proposals approved by the Institutional Review Board at the VCU TDAAC.

### Isolation and Culture of Primary Human Astrocyte and Normal Immortal Prostate Epithelial Stem Cells

Primary normal human astrocytes and normal immortal prostate epithelial cells were cultured in ultra-low attachment plates and flasks (Corning) in Clonetics EBM media and Keratinocyte-SFM media (Gibco, USA), respectively. The cells were stained with CD44 and alpha2beta1 integrin antibody, sorted and cultured further under ultra-low attachment conditions.

### Self-Renewal Assay

Sphere-forming assays were used to determine clonogenic growth potential in vitro of both normal and neoplastic cells (27). Sorted GSCs and NSGC populations were diluted to a density of 500 cells/ml. 2 μl of the diluted cell suspension was plated per well in a 96 well ultra-low attachment plate (Corning Inc., Corning, N.Y., USA), and 150 μl of serum-free medium was added, cultures were then observed daily (n=96). Additionally, flow cytometry with CD44 and CD133 antibody (Miltenyi Biotech) was performed to assess the stem populations.

### Gene Expression Arrays, Protein Expression Arrays and Analyses

TissueScan Brain Cancer Tissue cDNA array I, containing 46 malignant (covering four stages) and 2 tumor-adjacent normal tissue cDNAs, were obtained from Origene Technologies, (Rockville, Md., USA). This array was analyzed for mda-9, c-myc, Oct4 and Sox2 expression using taqman probes (Invitrogen) according to the manufacturer's protocol. A human CSC array (Qiagen) was used according to the manufacturer's protocol to analyze a clinical GBM sample VG2 (shcon) and an mda-9 kd clone of VG2 (shmda-9). A human angiogenesis antibody array (R&D systems) was used to analyze the conditioned media from shcon and shmda-9 CSCs. 84 genes were studied. The data was analyzed on the Qiagen web-based PCR array data analysis software.

### Analysis of Human Angiogenesis Proteins

Human angiogenesis antibody array (R&D systems) were used to analyze the conditioned media from shcon and shmda-9 DU-145 and MDA-MB-231 CSCs.

### Promoter Reporter Assay

Luciferase reporter assays were performed using 2×10⁵ cells infected with either Ad.5/3.shcon or Ad.5/3.shmda-9. Twenty-four hours post-infection, cells were transfected with an RBPJK luciferase reporter construct with Lipofectamine 2000 as described (21). Cell lysates were harvested and luciferase activity was measured using a Dual-Luciferase Reporter Assay system (Promega) according to the manufacturer's instructions. Luciferase activity was normalized to Renilla activity, and data represent the average of triplicates±S.D.

### Reverse Transcription Polymerase Chain Reaction

Total RNA was isolated by TRIzol extraction (Invitrogen) and purified using the RNeasy kit (Qiagen). First-strand cDNA was synthesized with SuperScript III reverse transcriptase (Invitrogen). Quantitative PCR for KRT20, ANPEP and PRSS7 were carried out by using the TagMan Gene expression assays (Invitrogen), and were normalized to 18S expression (Invitrogen). Probes details are as follows:

| | | | |
|---|---|---|---|
| mda-9 | Hs01045460_g1 | myc | Hs00153408_m1 |
| Nanog | Hs04399610_g1 | Sox2 | Hs00415716_m1 |
| Oct4 | Hs04260367_gH | CD133 | Hs01009250_m1 |
| Notch1 | Hs01062014_m1 | 18S | Hs99999901_s1 |

### Western Blotting

Cells were lysed on ice in lysis buffer (20 mM Tris-HCl (pH 7.5), 150 mM NaCl, 1 mM Na₂EDTA, 1 mM EGTA, 1% Triton-100, 2.5 mM Sodium pyrophosphate, 1 mM β-glycerophosphate, 1 mM Na₃VO₄, 1 μg/ml Leupeptin). Protein samples were prepared after protein concentration was determined, and were loaded onto 8% SDS-PAGE for immunoblotting detection. For densitometric evaluation, X-ray films were scanned and analyzed with Image software (National Institutes of Health [NIH]).

### Antibodies

MDA-9 Abnova (H00006386-M01) (western blot)

C-MYC Abcam (ab32072) (western blot)

STAT-3 (Flow cytometry) (western blot)

P-STAT-3 CST ((Y705) (M9C6) #9145 (Flow cytometry) (western blot)

P44/42 (Flow cytometry) (western blot)

P-p44/42(Flow cytometry) (western blot)

NOTCH1-PE, BD Pharmingen (MHN1-519) (Flow cytometry)

DLL1-APC Miltenyi Biotec (clone: MHD1-314) (Flow cytometry)

Numb Abcam (ab123891) (Flow cytometry)

SRC CST #2108 (western blot)

p-SRC BD (560094) (Flow cytometry) CST #6943 (western blot)

CD44-PE BD Pharmingen (550989) (Flow cytometry)

CD24-FITC BD Pharmingen (555427) (Flow cytometry)

CD133-APC Miltenyi Biotec (130-090-826) (Flow cytometry)

Alpha2 beta1 integrin Abcam ((ab30483) (Flow cytometry)

SOX2, CST (#3579) (immunofluorescence)

OCT4, CST (#2840) (immunofluorescence)

### Immunofluorescent Staining

SOX2, OCT4 staining was performed according to the manufacturer's instructions (CST), followed by imaging by laser confocal microscopy (Leica). The images were analyzed by Zen software.

### Tumorigenicity Studies

All experiments and procedures involving mice were approved by the Institutional Animal Care and Use Committee of Virginia Commonwealth University. For the intracranial brain tumor model, athymic female NCr-nu/nu mice (National Cancer Institute—Frederick) were used (n=10 per group). Mice were maintained under pathogen-free conditions in facilities approved by the American Association for Accreditation of Laboratory Animal Care and in accordance with current regulations and standards of the US Department of Agriculture, the US Department of Health and Human Services, and the NIH. Mice were anesthetized through i.p. administration of ketamine (40 mg/kg) and xylazine (3 mg/kg) and immobilized in a stereotactic frame (Stoelting). Intracerebral injections of 1.5×10⁴ cells in 2 μL per mouse were done using an automated injector (Stoelting) as described earlier (21). Tumor burden was determined by bioluminescent imaging. For DU-145 and MDA-MB-231 xenografts, 1×10⁵ CSCs were implanted subcutaneously into the right flanks of athymic male and female NCr-nu/nu mice, respectively. Tumor burden was determined by tumor size and weight.

For DU-145 and MDA-MB-231 xenografts, 1×10⁵ cells were implanted subcutaneously into the right flanks of athymic nude mice as described previously (24, 28). For intra-tumoral injections, intratumoral injections of Ad.5/3-vec or Ad.5/3-mda-9 were given to the tumors at a dose of 1×10⁸ v.p. in 100 μL, after establishing visible tumors of ˜100 mm³. The injections were given 3 times the first week and then 2 times/wk for two more weeks for a total of seven injections. Tumor burden was determined by bioluminescent imaging (28).

Animals of each group were monitored until they reached the point of euthanization according to the VCU-IACUC approved protocol and survival data was analyzed.

### Histology

Mice were euthanized according to the veterinarian's suggestions (approximately 3 months from intracranial injection). The mice were carefully dissected to obtain the brain tissue. Paraffin-embedded tissues were sectioned at 4-μm thickness and stained with Haematoxylin and Eosin.

### In Vivo Metastasis Studies

Luciferase-labeled CSCs were delivered to athymic nude mice via intracardiac injection as described previously (29). Luciferase-labeled CSC shcon and shmda-9 cells (1×10⁶) were delivered via intracardiac injection. The mice were continuously monitored for weight and physiological symptoms. 30 days post injection, D-luciferin was injected (150 mg Luciferin/body weight). Luciferase activity was used to assess relative tumor burden by bioluminescence imaging (28).

### CAM Assay

Chicken chorioallantoic membrane (CAM) assays were performed using 9-day-old chick embryos; cells were seeded on the CAM surface according to established protocols (21). One week after inoculation, the neovasculature was examined and photographed.

### Angiogenesis Array

Equal amounts of protein (500 μg) in 100-μL samples of conditioned media were assayed using human angiogenesis antibody arrays (R&D Biosystems) and quantified according to the manufacturer's instructions.

### Flow Cytometry Sorting and Analysis

CD44, CD24, CD133, alpha2beta1 integrin, NOTCH1, DLL1, STAT-3, p-STAT-3, p44/42, p-p44/42 staining and Annexin V staining were performed according to the manufacturer's instructions, followed by flow cytometric analysis using BD DIVA.

### ELISA

CXC1-16 ELISA kit (R&D Systems) was used according to manufacturer's protocol to analyze conditioned media with normalized protein content.

### Intracellular Flow Cytometry

STAT3, p-STAT3, p44/42, p-p44/42, p-Src, and Numb proteins were assessed by intra-cellular flow cytometry (30, 31). Cell fixation, permeabilization and antibody staining were performed according to the manufacturer's instructions, followed by flow cytometry analysis using BD DIVA.

### Peptide Blocking/Activation Studies

1×10⁵ control and treated CSCs were cultured in 6-well ultra-low attachment plates. NOTCH-1 blocking peptide (Biovision) and DLL1 peptide (Abcam) were used at a concentration of 10 μg/ml and incubated for 48 hours. After incubation, the cells were stained and analyzed for viability, spheroid size and structure.

### Live/Dead Assay

Live/Dead staining was performed according to the manufacturer's instructions (Invitrogen), followed by imaging by laser confocal microscopy (Leica). The images were analyzed by Zen software.

shRNA Knockdown

shRNA sequences were obtained through Qiagen with the following sequences:

[SEQ No. 1]

5′-TTGACTCTTAAGATTATGTAA-3′ (shmda-9 #3)
and

[SEQ No. 2]

5′-TGGGATGGTCTTAGAATATTT-3′ (shmda-9 #4).

Ad.5/3.shmda-9 was constructed as previously described (21) using the following primer sequences:

forward:

[SEQ No. 3]

5′GCCTGCTTTTATCTTTGAACATATTATTAAGCGAATGAAGCCTAGTAT
AATGAAAA GCCTAATGGACCACACCATTCCTGAG-3′
and
reverse:

[SEQ No. 4]

3′-CGGACGAAAATAGAAACTTGTATAATAATTCGCTTACTTCGGATCAT
ATTACTTTTCGGATTACCTGGTGTGGT AAGGACTC-5′.

The cells were infected with Ad. 5/3.shcon and Ad.5/3.shmda-9 (1000 v.p./cell) in serum free media for 4 hours and the media was replaced with fresh complete media.

### Overexpression Studies

The genomic sequence of mda-9/syntenin was amplified by PCR using genomic DNA as template and primers, sense: 5′-CTGCAAAAATGTCTCTCTATCC-3′ [SEQ No. 5] and anti-sense: 5′-GGTGCCGTGAATTTTAAACCTCAG-3′ [SEQ No. 6]. The PCR product was cloned into a pREP4 expression vector and then it was digested and released with Xho and BamH1 and subcloned into the pcDNA3.1 (+hygro) plasmid (Invitrogen). This plasmid was used to overexpress mda-9 in RwPE-1 cells. Additionally, this plasmid was transfected into CSCs by incubating with Fugene 6 (overnight shaking) and then replaced with fresh complete media.

The DNA fragment (990-bp) having the mda-9/syntenin gene was isolated from plasmid p0tg-CMV-MDA-9 (21) and cloned between BglII and EcoRV sites downstream of the cytomegalovirus (CMV) promoter in the plasmid pSh-CMV. The shuttle plasmids were recombined with genomic DNA of Ad.5/3.Luc1 vector as previously described (21) to derive plasmids pAd.5/3.shmda-9 or pAd.5/3.mda-9. The resultant plasmids were digested with Pad to release the recombinant adenovirus genomes and then transfected into human embryonic kidney (HEK)-293 cells to rescue the corresponding Ad.5/3-based vectors. The rescued viruses were amplified using HEK-293 cells and purified by cesium chloride double ultracentrifugation using standard protocol, and the titers of infectious viral particles were determined by plaque assay using HEK-293 cells as described (21). The cells were infected with Ad.5/3.mda-9 (1000 viral particles/cell) in incomplete media for 4 hours that was then replaced with fresh complete media.

pcDNA3-c-myc plasmid (Addgene #16011), pCMV human p27 (Addgene #14049), EF.STAT-3C.Ubc.GFP (Addgene #24983) were used for forced expression of c-myc, p27 and CA-Stat-3 respectively. The CA-Src and Src plasmid were kind gifts from Dr. Jeffrey N. Bruce. The plasmids were transfected into CSCs by incubating with Fugene 6 according to the manufacturer's instructions, under overnight shaking, and then replaced with fresh complete media.

### Statistical Analysis

For all in vitro and ex vivo experiments, statistical analyses were conducted using Student's t test (Microsoft Excel). For in vivo studies, statistical analyses were performed using Kaplan-Meier method (survival studies), chi-square test (Microsoft Excel) (tumor incidence), and Mann Whitney-U test (number of metastatic sites and tumor burden). Pearson's correlation coefficient (R) and coefficient of determination (R²) were calculated for correlation analysis. The data from clinical samples were analyzed using Microsoft Excel's multiple regression analysis tool. All statistical tests were two-sided, and p values ≤0.05 and ≤0.01 were considered to be significant and highly significant, respectively. Patient data was analyzed using correlation heatmap and cluster analysis tools (Plotly Technologies Inc. Montreal, QC). The RT² Profiler PCR Array Data Analysis software was used to study the statistical significance of cancer stem cell array data, and a minimum of an ˜4-fold decrease was analyzed by selecting the statistical boundary for Log₁₀ shmda-9 del del CT/Log₁₀ shcon del del CT as 4.

### REFERENCES

1. Fisher P B, and Weinstein I B. Chemical-viral interactions and multistep aspects of cell transformation. IARC Sci Publ 1980; 27:113-31.
2. Pattabiraman D R, and Weinberg R A. Tackling the cancer stem cells—what challenges do they pose? Nature Rev Drug Discov 2014; 13:497-512.
3. Li Y, Rogoff H A, Keates S, Gao Y, Murikipudi S, Mikule K, et al. Suppression of cancer relapse and metastasis by inhibiting cancer stemness. Proc Natl Acad Sci USA 2015; 112:1839-44.
4. Talukdar S, and Kundu S C. Engineered 3D silk-based metastasis models: interactions between human breast adenocarcinoma, mesenchymal stem cells and osteoblast-like cells. Adv Funct Mater 2013; 23:5249-60.
5. Diehn M, and Majeti R. Metastatic cancer stem cells: an opportunity for improving cancer treatment? Cell Stem Cell 2010; 6:502-3.
6. Al-Hajj M, Wicha M S, Benito-Hernandez A, Morrison S J, and Clarke M F. Prospective identification of tumorigenic breast cancer cells. Proc Natl Acad Sci USA 2003; 100:3983-8.
7. Singh S K, Hawkins C, Clarke I D, Squire J A, Bayani J, Hide T, et al. Identification of human brain tumour initiating cells. Nature 2004; 432:396-401.
8. Visvader J E, and Lindeman G J. Cancer stem cells in solid tumours: accumulating evidence and unresolved questions. Nature Rev Cancer 2008; 8:755-68.
9. Lahad J P, Mills G B, and Coombes K R. Stem cell-ness: a “magic marker” for cancer. J Clinical Invest 2005; 115:1463-7.
10. Kegelman T P, Hu B, Emdad L, Das S K, Sarkar D, and Fisher P B. In vivo modeling of malignant glioma: the road to effective therapy. Adv Cancer Res 2014b; 121:261-330.
11. Mao P, Joshi K. Li J, Kim S H, Li P, Santana-Santos L, et al. Mesenchymal glioma stem cells are maintained by activated glycolytic metabolism involving aldehyde dehydrogenase 1A3. Proc Natl Acad Sci USA 2013; 110:8644-9.
12. Medema J P. Cancer stem cells: the challenges ahead. Nature Cell Biol 2013; 15: 338-44.
13. Collins A T, Habib F K, Maitland N J, and Neal D E. Identification and isolation of human prostate epithelial stem cells based on alpha(2)beta(1)-integrin expression. J Cell Sci 2001; 114: 3865-72.
14. Pan G, and Thomson J A. Nanog and transcriptional networks in embryonic stem cell pluripotency. Cell Res 2007; 17:42-9.
15. Fouse S D, and Costello J F. Cancer Stem Cells Activate STAT3 the EZ Way. Cancer Cell 2013; 23:711-13.
16. Zhao D, Mo Y, Li M T, Zou S W, Cheng Z, Sun Y P, Xiong Y. Guan K L, and Lei Q Y. NOTCH-induced aldehyde dehydrogenase 1A1 deacetylation promotes breast cancer stem cells. J Clin Invest 2014; 124:5453-65.
17. Lee T K, Castilho A, Cheung V C, Tang K H, Ma S, and Ng, 10. CD24(+) liver tumor-initiating cells drive self-renewal and tumor initiation through STAT3-mediated NANOG regulation. Cell Stem Cell 2011; 9:50-63.
18. Nair R, Roden D L, Teo W S, McFarland A, Junankar S. Ye S, et al. c-Myc and Her2 cooperate to drive a stem-like phenotype with poor prognosis in breast cancer. Oncogene 2014; 33:3992-4002.
19. Schmidt C. Cancer stem cells in the crosshairs. Cancer Discov 2012; 5:384.
20. Sarkar D, Boukerche H, Su Z Z, and Fisher P B. mda-9/Syntenin: more than just a simple adapter protein when it comes to cancer metastasis. Cancer Res 2008; 68:3087-93.
21. Kegelman T P, Das S K, Hu B, Bacolod M D, Fuller C E, Menezes M E, et al. MDA-9/syntenin is a key regulator of glioma pathogenesis. Neuro Oncol 2014a; 16: 50-61.
22. Boukerche H, Su Z Z, Emdad L, Baril P, Balme B, Thomas L, et al. mda-9/Syntenin: a positive regulator of melanoma metastasis. Cancer Res 2005; 65:10901-11.
23. Das S K, Bhutia S K, Sokhi U K, Azab B, Su Z Z, Boukerche H, et al. Raf kinase inhibitor RKIP inhibits MDA-9/syntenin-mediated metastasis in melanoma. Cancer Res 2012; 72:6217-26.
24. Qian X L, Li Y Q, Yu B, Gu F, Liu F F, Li W D, et al. Syndecan binding protein (SDCBP) is overexpressed in estrogen receptor negative breast cancers, and is a potential promoter for tumor proliferation. PloS one 2013; 8:e60046.
25. Bilodeau M, Sauvageau G. Uncovering stemness. Nat Cell Biol. 2006; 8: 1048-1049.
26. Xu Q, Yuan X, Tunici P, Liu G, Fan X, Xu M, et al. Isolation of tumor stem-like cells from benign tumors. Br J Cancer 2009; 101:303-11.
27. Cao L, Zhou Y, Zhai B, Liao J, Xu W, Zhang R, Li J, Zhang Y, Chen L, Qian H, Wu M, Yin Z. Sphere-forming cell subpopulations with cancer stem cell properties in human hepatoma cell lines. BMC Gastroenterol. 2011; 11:71.
28. Dash R, Azab B, Quinn B A, Shen X, Wang X Y, Das S K, et al. Apogossypol derivative BI-97C1 (Sabutoclax) targeting Mcl-1 sensitizes prostate cancer cells to mda-7/IL-24-mediated toxicity. Proc Natl Acad Sci USA 2011; 108:8785-90.
29. Dasgupta S, Menezes M E, Das S K, Emdad L, Janjic A, Bhatia S, et al. Novel role of MDA-9/syntenin in regulating urothelial cell proliferation by modulating EGFR signaling. Clin Cancer Res 2013; 19:4621-33.
30. Perez O D, Nolan G P. Simultaneous measurement of multiple active kinase states using polychromatic flow cytometry. Nat Biotechnol. 2002; 20: 155-162
31. Krutzik P O, Nolan G P. Intracellular phospho-protein staining techniques for flow cytometry: monitoring single cell signaling events. Cytometry A. 2003; 55: 61-70.
32. Li Y, Ye X, Tan C, Hongo J A, Zha J, Liu J, Kallop D, Ludlam M J, and Pei L. Axl as a potential therapeutic target in cancer: role of Axl in tumor growth, metastasis and angiogenesis. Oncogene 2009; 28:3442-55.
33. Niwa H, Burdon T, Chambers I, Smith A. Self-renewal of pluripotent embryonic stem cells is mediated via activation of STAT3. Genes & Development. 1998; 12: 2048-2060.
34. Sherry M M, Reeves A, Wu J K, Cochran B H. STAT3 is required for proliferation and maintenance of multipotency in glioblastoma stem cells. Stem Cells. 2009; 27: 2383-2392.
35. Takebe N, Harris P J, Warren R Q, and Ivy S P. Targeting cancer stem cells by inhibiting Wnt, Notch, and Hedgehog pathways. Nature Rev Clinical Oncol 2011; 8:97-106.
36. Carro M S, Lim W K, Alvarez M J, Bollo R J, Zhao X, Snyder E Y et al. The transcriptional network for mesenchymal transformation of brain tumours. Nature 2010; 463:318-25.
37. Gabay M, Li Y, and Felsher D W. MYC activation is a hallmark of cancer initiation and maintenance. Cold Spring Harb Perspect Med 2014; 4: a014241.
38. Wang J, Wang H, Li Z, Wu Q, Lathia J D, McLendon R E, Hjelmeland A B, Rich J N. c-Myc is required for maintenance of glioma cancer stem cells. PLoS One. 2008; 3: e3769.
39. Laurenti E, Varnum-Finney B, Wilson A, Ferrero I, Blanco-Bose W E, Ehninger A, Knoepfler P S, Cheng P F, MacDonald H R, Eisenman R N, Bernstein I D, Trumpp A. Hematopoietic stem cell function and survival depend on c-Myc and N-Myc activity. Cell Stem Cell. 2008; 3: 611-624.
40. Lathia J D, Mack S C, Mulkearns-Hubert E E, Valentim C L L, Rich J N. Cancer stem cells in glioblastoma. Genes & Development. 2015; 29: 1203-1217.
41. Altaner C. Glioblastoma and stem cells. Neoplasma. 2008; 55: 369-374.
42. Ortensi B, Setti M, Osti D, Pelicci G. Cancer stem cell contribution to glioblastoma invasiveness. Stem Cell Research & Therapy. 2013; 4: 18.
43. Tkach M, Rosemblit C, Rivas M A, Proietti C J, Diaz Flaque M C, Mercogliano M F, et al. p42/p44 MAPK-mediated Stat3Ser727 phosphorylation is required for progestin-induced full activation of Stat3 and breast cancer growth. Endocr Relat Cancer 2013; 20:197-212.
44. Turkson J, Bowman T, Garcia R, Caldenhoven E, De Groot R P, Jove R. Stat3 activation by Src induces specific gene regulation and is required for cell transformation. Mol Cell Biol. 1998; 18: 2545-2552.
45. Plaza-Menacho I, van der Sluis T, Hollema H, Gimm O, Buys C H, Magee A I, Isacke C M, Hofstra R M, and Eggen B J. Ras/ERK1/2-mediated STAT3 Ser727 phosphorylation by familial medullary thyroid carcinoma-associated RET mutants induces full activation of STAT3 and is required for c-fos promoter activation, cell mitogenicity, and transformation. J Biol Chem 2007; 282:6415-24.
46. Zhao D, Pan C, Sun J, Gilbert C, Drews-Elger K, Azzam D J, Picon-Ruiz M, Kim M, Ullmer W, El-Ashry D, Creighton C J, Slingerland J M. VEGF drives cancer-initiating stem cells through VEGFR-2/Stat3 signaling to upregulate Myc and Sox2. Oncogene. 2015; 34: 3107-3119.
47. Zhang W, Zong C S, Hermanto U, Lopez-Bergami P, Ronai Z, and Wang L H. RACK I recruits STAT3 specifically to insulin and insulin-like growth factor 1 receptors for activation, which is important for regulating anchorage-independent growth. Mol Cell Biol 2006; 26:413-24.
48. Boukerche H, Su Z Z, Prevot C, Sarkar D, and Fisher P B. mda-9/Syntenin promotes metastasis in human melanoma cells by activating c-Src. Proc Natl Acad Sci USA 2008; 105:15914-19.
49. Gonzalez M E, Moore H M, Li X, Toy K A, Huang, W, Sabel M S, et al. EZH2 expands breast stem cells through activation of NOTCH1 signaling. Proc Natl Acad Sci USA 2014; 111: 3098-103.
50. McGill M A, Dho S E, Weinmaster G, and McGlade C J. Numb regulates post-endocytic trafficking and degradation of Notch1. J Biol Chem 2009; 284:26427-38.
51. Weiss A, Baumgartner M, Radziwill G, Dennler J, and Moelling K. c-Src is a PDZ interaction partner and substrate of the E3 ubiquitin ligase Ligand-of-Numb protein X1. F E B S Lett 2007; 581:5131-6.
52. Adam M G, Berger C, Feldner A, Yang W J, Wustehube-Lausch J, Herberich S E, et al. Synaptojanin-2 binding protein stabilizes the Notch ligands DLL1 and DLL4 and inhibits sprouting angiogenesis. Circulation Res 2013; 113:1206-18.
53. Heuss S F, Ndiaye-Lobry D, Six E M, Israel A, and Logeat F. The intracellular region of Notch ligands Dll1 and Dll3 regulates their trafficking and signaling activity. Proc Natl Acad Sci USA 2008; 105:11212-7.
54. Estrach S, Legg J, and Watt F M. Syntenin mediates Delta1-induced cohesiveness of epidermal stem cells in culture. J Cell Sci 2007; 120:2944-52.
55. Okumura F, Yoshida K, Liang F, and Hatakeyama S. MDA-9/syntenin interacts with ubiquitin via a novel ubiquitin-binding motif. Mol Cell Biochem 2011; 352:163-72.
56. Palomero T, Lim W K, Odom D T, Sulis M L, Real P J, Margolin A, et al. NOTCH1 directly regulates c-MYC and activates a feed-forward-loop transcriptional network promoting leukemic cell growth. Proc Natl Acad Sci USA 2006; 103:18261-6.
57. Wang H, Zang C, Taing L, Arnett K L, Wong Y J, Pear W S, et al. NOTCH1-RBPJ complexes drive target gene expression through dynamic interactions with superenhancers. Proc Natl Acad Sci USA 2014; 111:705-10.
58. Cole M D. MYC association with cancer risk and a new model of MYC-mediated repression. Cold Spring Harb Perspect Med 2014; 4: a014316.
59. Amati B I, Alevizopoulos K, Vlach J. Myc and the cell cycle. Front Biosci 1998; 3:d250-68.
60. Nickeleit I, Zender S, Kossatz U, and Malek N P. p27kip 1: a target for tumor therapies? Cell Division 2007; 2:13.
61. Li H, Collado M, Villasante A, Matheu A, Lynch C J, Canamero M, et al. p27(Kip1) directly represses Sox2 during embryonic stem cell differentiation. Cell Stem Cell 2012; 11:845-852.
62. Chen S, Li X, Lu D, Xu Y, Mou W, Wang L, et al. SOX2 regulates apoptosis through MAP4K4-survivin signaling pathway in human lung cancer cells. Carcinogenesis 2014; 35: 613-23.
63. Liu G, Yuan X, Zeng Z, Tunici P, Ng H, Abdulkadir I R, et al. Analysis of gene expression and chemoresistance of CD133+ cancer stem cells in glioblastoma. Mol Cancer 2006; 5:67.
64. Talukdar S, Emdad L. Das S K, Sarkar D, Fisher P B. Evolving strategies for therapeutically targeting cancer stem cells (CSCs). Adv Cancer Res. 2016; in press.
65. Kim C F, Dirks P B. Cancer and stem cell biology: how tightly intertwined. Cell Stem Cell 2008; 3 2:147-150.

1-19. (canceled)

20. A method of treating a cancer in a subject in need thereof, the method comprising administering to said subject an effective amount of an agent that decreases expression of MDA-9/Syntenin (SDCBP), wherein said cancer is a therapy resistant cancer.

21. The method of claim 20, wherein said therapy resistant cancer is a chemoresistant cancer.

22. The method of claim 20, wherein the agent is a small interfering RNA (siRNA) or a short hairpin RNA (shRNA) comprising a sequence specific for the gene of MDA-9/Syntenin (SDCBP) or using CRSIPR/Cas9 or similar genome targeted editing approach.

23. The method of claim 20, wherein the decrease in expression of MDA-9/Syntenin (SDCBP) is achieved by CRSIPR/Cas9 or other genome targeted editing techniques.

24. The method of claim 20, wherein the decrease in the expression of MDA-9/Syntenin (SDCBP) decreases the expression of c-Myc through the regulation of the NOTCH1 pathway.

25. The method of claim 20, wherein the cancer is selected from the group consisting of prostate cancer, breast cancer, gastric cancer, lung cancer, brain cancer, pancreatic cancer and neuroblastoma.

26. The method of claim 25, wherein the brain cancer is glioblastoma.

27. A method of treating cancer in a subject in need thereof, the method comprising administering to said subject an effective amount of an agent that decreases expression of MDA-9/Syntenin (SDCBP), wherein said subject has previously received a cancer treatment or is currently receiving cancer treatment.

28. The method of claim 27, wherein said administering occurs simultaneously with said cancer treatment.

29. The method of claim 27, wherein said administering occurs following said cancer treatment.

30. The method of claim 27, wherein the agent is a small interfering RNA (siRNA) or a short hairpin RNA (shRNA) comprising a sequence specific for the gene of MDA-9/Syntenin (SDCBP) or using CRSIPR/Cas9 or similar genome targeted editing approach.

31. The method of claim 27, wherein the decrease in expression of MDA-9/Syntenin (SDCBP) is achieved by CRSIPR/Cas9 or other genome targeted editing techniques.

32. The method of claim 27, wherein the decrease in the expression of MDA-9/Syntenin (SDCBP) decreases the expression of c-Myc through the regulation of the NOTCH1 pathway.

33. The method of claim 27, wherein the cancer is selected from the group consisting of prostate cancer, breast cancer, gastric cancer, lung cancer, brain cancer, pancreatic cancer and neuroblastoma.

34. The method of claim 33, wherein said brain cancer is glioblastoma.

35. The method of claim 27, wherein said decrease in the expression of MDA-9/Syntenin (SDCBP) selectively kills cancer stem cells.
